(19) United States
(12) Patent Application Publication
(10) Pub. No.: US 2022/0233821 A1
(43) Pub. Date: Jul. 28, 2022

(54) DISTAL STABILIZER FOR USE FOR CATHETER DELIVERY IN BIOLOGICAL LUMEN, DELIVERY SYSTEM FOR DELIVERING TREATMENT DEVICE, AND TREATMENT DEVICE

(71) Applicant: Bolt Medical Inc., Tokyo (JP)

(72) Inventor: Naoki INUZUKA, Tokyo (JP)

(73) Assignee: Bolt Medical Inc., Tokyo (JP)

(21) Appl. No.: 17/657,293

(22) Filed: Mar. 30, 2022

Related U.S. Application Data
(63) Continuation of application No. PCT/JP2021/016090, filed on Apr. 20, 2021.

(30) Foreign Application Priority Data
Jan. 26, 2021 (JP) 2021-010388

(51) Int. Cl. A61M 25/04; A61M 25/09

(52) U.S. Cl. CPC A61M 25/04; A61M 2025/09183; A61M 25/09

(57) ABSTRACT

A distal stabilizer for use for catheter delivery in a biological lumen, the distal stabilizer comprising: a thread-like delivery member; and a locking device that extends from a distal end of the thread-like delivery member and is capable of locking onto an inner wall of the biological lumen with an expanding force. The distal stabilizer is usable in a catheter delivery operation including: releasing the locking device, which has been loaded in a first catheter while being in a reduced diameter state, from a distal end of the first catheter, thereby causing the locking device to lock onto the inner wall; and advancing a catheter including a second catheter having an inner diameter larger than the first catheter toward a distal side while the locking device remains locked on the inner wall. The distal stabilizer is configured such that after completion of the catheter delivery operation, the locking device is reduced in diameter to be resheathed into one of the catheters, and the distal stabilizer is removed out of the biological lumen.

### TECHNICAL FIELD

The present invention relates to a distal stabilizer for use for catheter delivery in a biological lumen, a delivery system for delivering a treatment device in a biological lumen, and a treatment device.

### BACKGROUND ART

Certain treatments are administered by means of a catheter introduced in a biological lumen, such as a patient's artery, and having a distal end placed in a vicinity of a target position. The inner path of the catheter is used to deliver a treatment device to the target position, or the catheter itself is used as a treatment device.

Such a catheter needs to have an inner diameter (passage diameter) that is sufficiently large to allow insertion of a treatment device or to enable the catheter itself to function as a treatment device. An operation for placing such a relatively large catheter (purpose catheter) in a vicinity of a target position in a biological lumen is usually facilitated by using tools, such as a catheter (microcatheter) thinner than the purpose catheter and a delivery wire (which is sometimes called a guidewire or a pusher wire) inserted into the catheter are usually used. This operation typically includes causing the delivery wire to precede the catheters, guiding the microcatheter externally fitted over the delivery wire to the vicinity of the target position, and then, guiding the purpose catheter externally fitted over the microcatheter to the vicinity of the target position.

For example, if the target position is at the end of a tortuous blood vessel with many twists and turns, the delivery wire unavoidably has to be introduced to reach a point located farther distally than necessary such that the delivery wire can resist a force applied when the relatively rigid purpose catheter passes through the tortuous vessel. However, this operation can carry, for example, the risk of perforation of the blood vessel, the risk of straying of the delivery wire into a perforating branch (a small bifurcated artery that is not the target position), and the risk of bleeding due to perforation of the perforating branch. For these reasons, the target positions to be treated by this manipulation have been restricted to relatively proximal areas of biological lumens.

Patent Document 1 discloses an anchor device including a delivery wire and a stent joined to the leading end of the delivery wire. When the stent is extruded from a microcatheter and expanded, the delivery wire is anchored onto an inner wall of a blood vessel. This can mitigate some of the above risks.

Patent Document 1 discloses a catheter system having a structure in which three or more catheters are inserted into each other in a telescopic fashion. The thinnest catheter is first introduced into a blood vessel, and the second thinnest catheter externally fitted over the thinnest catheter is advanced with respect to the thinnest catheter. The third thinnest catheter externally fitted over the second thinnest catheter is advanced with respect to the second thinnest catheter. These processes are repeated, whereby the thickest catheter is delivered to a vicinity of a target position.
Patent Document 1: U.S. Pat. No. 9,682,216

### DISCLOSURE OF THE INVENTION

### Problems to be Solved by the Invention

Regarding the structure of the stent, Patent Document 1 illustrates a closed cell structure in which the vertexes of all zigzag wires are welded to each other. However, a stent with the closed cell structure is likely to kink in, for example, a tortuous blood vessel with many twists and turns, and is difficult to lock sufficiently in such blood vessel.

On the other hand, Patent Document 1 also discloses that the stent may have an open cell structure in which some of the vertexes of the zigzag wires are left un-welded since the open cell structure makes it easy for the stent to follow sharp bends. However, it is technical common knowledge that the open cell structure makes it difficult for the stent to be resheathed into a catheter, as shown by the fact that the open cell structure has been mainly adopted into indwelling stents. In fact, the stent with the open cell structure disclosed in Patent Document 1 is extremely difficult to resheathe into a catheter.

In addition, in the case of telescopic catheter systems, such as the system disclosed in Patent Document 1, since the means for delivering a treatment device is limited to the thickest catheter, it is difficult to deliver a treatment device to many of target positions.

The present invention has been achieved in view of the foregoing circumstances. It is an object of the present invention to provide a distal stabilizer that has excellent performance in locking onto an inner wall of a biological lumen and can be easily resheathed into a catheter after use, to provide a treatment device delivery system including the distal stabilizer, and to provide a treatment device for use in combination with the delivery system.

It is another object of the present invention to provide a treatment device delivery system including a distal stabilizer that can lock onto an inner wall of a biological lumen and enable an increase in a degree of freedom of a target position to which a treatment device can be delivered, and to provide a treatment device for use in combination with the delivery system.

It is yet another object of the present invention to provide a treatment device delivery system including a distal stabilizer that can lock onto an inner wall of a biological lumen, the treatment device delivery system being capable of facilitating delivery of a treatment device to an aneurysm or a target position distal to the aneurysm, and to provide a treatment device for use in combination with the delivery system.

### Means for Solving the Problems

The present invention relates to a distal stabilizer for use for catheter delivery in a biological lumen. The distal stabilizer includes: a thread-like delivery member; and a locking device that extends from a distal end of the thread-like delivery member and is capable of locking onto an inner wall of the biological lumen with an expanding force. The distal stabilizer is usable in a catheter delivery operation including: releasing the locking device, which has been loaded in a first catheter while being in a reduced diameter state, from a distal end of the first catheter, thereby causing the locking device to lock onto the inner wall; and advancing a catheter including a second catheter having an inner diameter larger than the first catheter toward a distal side while the locking device remains locked on the inner wall. The distal stabilizer is configured such that after completion of the catheter delivery operation, the locking device is reduced in diameter to be resheathed into one of the catheters, and the distal stabilizer is removed out of the biological lumen.

According to the invention described above, the catheter including the second catheter may be advanced by way of pulling the delivery wirethread-like delivery member toward the proximal side while the locking stentlocking device remains locked on the inner wall.

According to the invention described above, the locking device may be released by way of retracting the first catheter toward the proximal side.

According to the invention described above, the locking device may fit with the first catheter having an inner diameter of 0.017 inches or less.

According to the invention described above, the locking device may be a locking stent including a body and an antenna via which a proximal end of the body converges at the thread-like delivery member, the body having open cells with protruding free ends that protrude only toward a distal side. The body may have a structure in which a plurality of cells are arranged helically with respect to a long axis direction of the locking stent.

According to the invention described above, the locking device may be a locking stent including a body and an antenna via which a proximal end of the body converges at the thread-like delivery member, the body having open cells with protruding free ends that protrude only toward a distal side. The locking stent may include one locking stent or two or more locking stents, and may have a total effective length of 5 mm or greater.

According to the invention described above, the locking device may have a radiopaque portion.

According to the invention described above, the locking device may be a locking stent including a body and an antenna via which a proximal end of the body converges at the thread-like delivery member, the body having open cells with protruding free ends that protrude only toward a distal side. The locking stent may have a filter that fills a gap over the entire circumferential direction of the body.

The present invention further relates to a delivery system for delivering a treatment device in a biological lumen. The delivery system includes: the distal stabilizer described above; and a plurality of catheters including the first and second catheters. One of the plurality of catheters that has an inner diameter larger than the first catheter functions as a purpose catheter. The delivery system is for use to enable delivery of the treatment device as the purpose catheter itself or the treatment device inserted in the purpose catheter to a target position in the biological lumen by way of placing the purpose catheter at the target position.

According to the invention described above, the target position may be located in an area having a blood vessel inner diameter of 0.5 mm or greater and 10 mm or less.

The present invention further relates to a delivery system for delivering a treatment device in a biological lumen. The delivery system includes: a distal stabilizer including a thread-like delivery member and a locking device, the locking device extending from a distal end of the thread-like delivery member, being capable of locking onto an inner wall of the biological lumen with an expanding force; and a plurality of catheters including a small catheter and a large catheter having an inner diameter larger than the small catheter. The delivery system is configured such that the locking device, which has been loaded in the small catheter while being in a reduced diameter state, is released from a distal end of the small catheter, thereby causing the locking device to lock onto the inner wall, and the small catheter is then removed out of the biological lumen while the locking device remains locked on the inner wall. A purpose catheter includes the large catheter, and the delivery system is for use to enable delivery of the treatment device as the purpose catheter itself or the treatment device inserted in the purpose catheter to a target position in the biological lumen by way of advancing the purpose catheter externally fitted over the thread-like delivery member toward a distal side and placing the purpose catheter at the target position.

The present invention further relates to a delivery system for delivering a treatment device in a biological lumen. The delivery system includes: a distal stabilizer including a thread-like delivery member and a locking device, the locking device extending from a distal end of the thread-like delivery member, being capable of locking onto an inner wall of the biological lumen with an expanding force; and a plurality of catheters including a small catheter and a large catheter having an inner diameter larger than the small catheter. A purpose catheter includes the large catheter, and the delivery system is for use to enable delivery of the treatment device as the purpose catheter itself or the treatment device inserted in the purpose catheter to a target position in the biological lumen, the delivery being performed by releasing, at a site distal to an aneurysm, the locking device, which has been loaded in the small catheter while being in a reduced diameter state, from a distal end of the small catheter, thereby causing the locking device to lock onto the inner wall, and by advancing the purpose catheter fitted over the thread-like delivery member toward the distal side and placing the purpose catheter at the target position in the biological lumen. The target position is the aneurysm or a position distal to the aneurysm.

According to the invention described above, the aneurysm may be a bifurcation aneurysm.

According to the invention described above, the delivery system may include a plurality of delivery systems for use in combination with each other. Each delivery system may be configured to lock onto an inner wall of an associated one of bifurcation vessels between which the bifurcation aneurysm resides, and each delivery system may be for use to deliver the treatment device as a flow diverter and to place the treatment device over an area from a location proximal to the bifurcation aneurysm to the bifurcation vessel.

According to the invention described above, the locking device may be a locking stent. The locking stent may consist only of closed cells or open cells, or may consist of a combination of the closed cells and the open cells.

According to the invention described above, the locking device may be a locking stent including a body and an antenna via which a proximal end of the body converges at the thread-like delivery member, the body having open cells with protruding free ends that protrude only toward a distal side. The locking stent may have a filter that fills a gap over the entire circumferential direction of the body.

According to the invention described above, the treatment device may be a thrombus suction device, a flow diverter, an aneurysm embolization device, a thrombus removal device, an aneurysm treatment stent, an intracranial artery stenosis treatment stent, a balloon catheter, a shunt, or a means for releasing a liquid embolic material.

The present invention further relates to a treatment device for use at a target position in a biological lumen, the treatment device being configured to be delivered by the delivery system.

According to the invention described above, the treatment device is used within the biological lumen or while projecting outside the biological lumen.

### Effects of the Invention

According to the invention described above, after delivery of a suction catheter as the treatment device to the target position, the locking device is caused to lock onto an inner wall of the biological lumen, an embolic material in the biological lumen is held between a distal end of the suction catheter and the locking device remaining locked on the inner wall, and the distal end of the suction catheter may be pressed onto the embolic material held between the distal end of the suction catheter and the locking device, and the embolic material may be suctioned while remaining held between the distal end of the suction catheter pressed onto the embolic material and the locking device remaining locked on the inner wall.

The distal stabilizer of the present invention has excellent performance in locking onto an inner wall of a biological lumen and is easy to resheathe into a catheter after use. The delivery system of the present invention includes the distal stabilizer that can lock onto an inner wall of a biological lumen and enables an increase in a degree of freedom of the target position to which the treatment device can be delivered.

### BRIEF DESCRIPTION OF THE DRAWINGS

FIG. 1 is a perspective view schematically illustrating a distal stabilizer 1 according to an embodiment of the present invention;

FIG. 2 is a perspective view illustrating a state in which a locking stent 3 of the distal stabilizer 1 is imaginarily fitted over a circular column-shaped object;

FIG. 3 is a developed view illustrating the locking stent 3 imaginarily developed on a plane;

FIG. 4 is a perspective view illustrating the locking stent 3 in a bent state;

FIG. 5 is a schematic diagram sequentially illustrating a therapeutic procedure that is performed using a delivery system 10 including the distal stabilizer 1;

FIG. 6 is a schematic diagram illustrating a different state of the therapeutic procedure of FIG. 5;

FIG. 7 is a schematic diagram illustrating a process subsequent to the process of FIG. 5;

FIG. 8 is a schematic diagram illustrating a process subsequent to the process of FIG. 7;

FIG. 9 is a schematic diagram illustrating a process subsequent to the process of FIG. 8;

FIG. 10 is a schematic diagram illustrating a process subsequent to the process of FIG. 9;

FIG. 11 is a schematic diagram illustrating a process subsequent to the process of FIG. 10;

FIG. 12 is a schematic diagram illustrating a process subsequent to the process of FIG. 11;

FIG. 13 is a schematic diagram illustrating a process subsequent to the process of FIG. 12;

FIG. 14 is a schematic diagram illustrating another embodiment of the process subsequent to the process of FIG. 9;

FIG. 15 is a schematic diagram illustrating a process subsequent to the process of FIG. 14;

FIG. 16 is a schematic diagram illustrating a process subsequent to the process of FIG. 15;

FIG. 17 is a schematic diagram illustrating a process subsequent to the process of FIG. 16;

FIG. 18 is a schematic diagram illustrating a process subsequent to the process of FIG. 17;

FIG. 19 is a schematic diagram illustrating another embodiment of the process subsequent to the process of FIG. 18;

FIG. 20 is a schematic diagram illustrating a process subsequent to the process of FIG. 19;

FIG. 21 is a schematic diagram illustrating a process subsequent to the process of FIG. 20;

FIG. 22 is a diagram illustrating a locking stent 3A having a filter 32;

FIG. 23 is a diagram illustrating a state in which a distal stabilizer is locked onto an inner wall of a biological lumen at a site distal to an aneurysm;

FIG. 24 is a schematic diagram illustrating a process subsequent to the process of FIG. 23;

FIG. 25 is a schematic diagram sequentially illustrating a treatment of a bifurcation aneurysm using a delivery system 10 including a distal stabilizer 1;

FIG. 26 is a schematic diagram illustrating a process subsequent to the process of FIG. 25;

FIG. 27 is a schematic diagram illustrating a process subsequent to the process of FIG. 26;

FIG. 28 is a schematic diagram illustrating a process subsequent to the process of FIG. 27;

FIG. 29 is a schematic diagram illustrating a process subsequent to the process of FIG. 28;

FIG. 30 is a schematic diagram illustrating a process subsequent to the process of FIG. 29;

FIG. 31 is a schematic diagram illustrating a process subsequent to the process of FIG. 30;

FIG. 32 is a schematic diagram illustrating a process subsequent to the process of FIG. 31;

FIG. 33 is a schematic diagram illustrating a process subsequent to the process of FIG. 32;

FIG. 34 is a schematic diagram illustrating a process subsequent to the process of FIG. 33; and

FIG. 35 is a schematic diagram illustrating a process subsequent to the process of FIG. 34.

FIG. 36 is a diagram (corresponding to FIG. 9) schematically illustrating a modification.

FIG. 37 is a diagram schematically illustrating an insufficient degree of contact according to the known art.

FIG. 38 is a diagram schematically illustrating an embodiment of the present invention overcoming the insufficient degree of contact illustrated in FIG. 37.

### PREFERRED MODE FOR CARRYING OUT THE INVENTION

Embodiments of a distal stabilizer 1 and a delivery system 10 of the present invention will be described below. Note that the drawings attached to the present specification are all schematic diagrams, and that for ease of understanding, the shape, the scale, and the aspect ratio, etc. of each part in each drawing are different from those of the original, or are illustrated with emphasis. In the present specification and the accompanying documents, terms specifying shapes, geometric conditions, and levels thereof, example of which include “parallel”, “orthogonal/perpendicular”, and “direction”, each encompass not only the strict meaning of the respective term, but also an extent which can be regarded as nearly parallel, nearly orthogonal/perpendicular, etc., and an extent which can be regarded as approximately being in the direction. In the present specification, a proximal side (proximal direction) that is close to a practitioner who performs a therapeutic procedure is denoted by the reference character D1, and a distal side (distal direction) that is distant from the practitioner is denoted by the reference character D2. An “X side” may also be referred to as an “X direction” as appropriate.

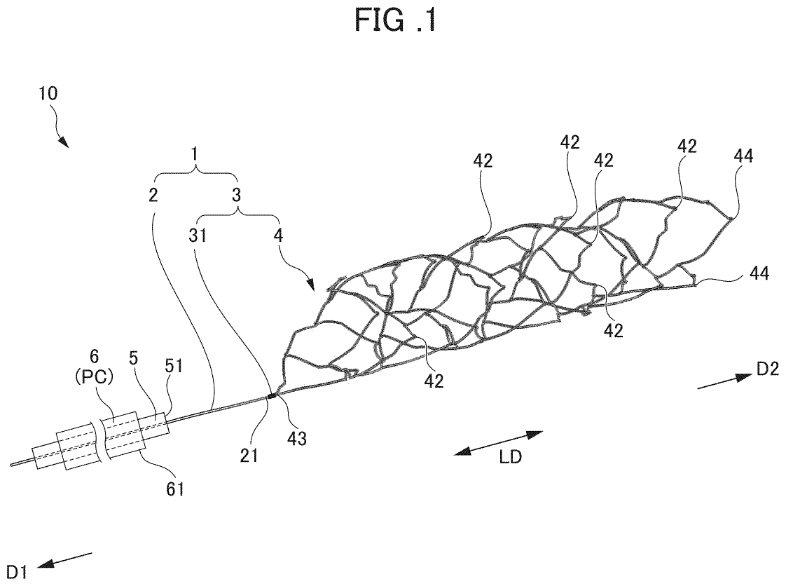

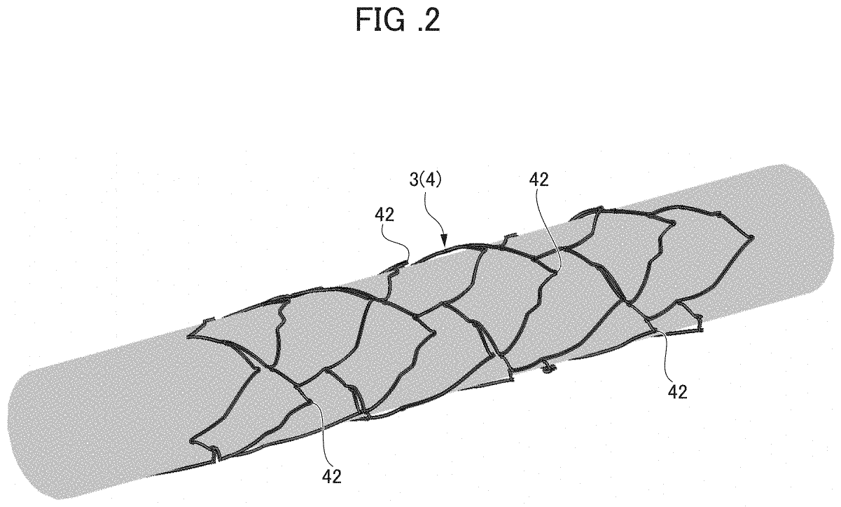

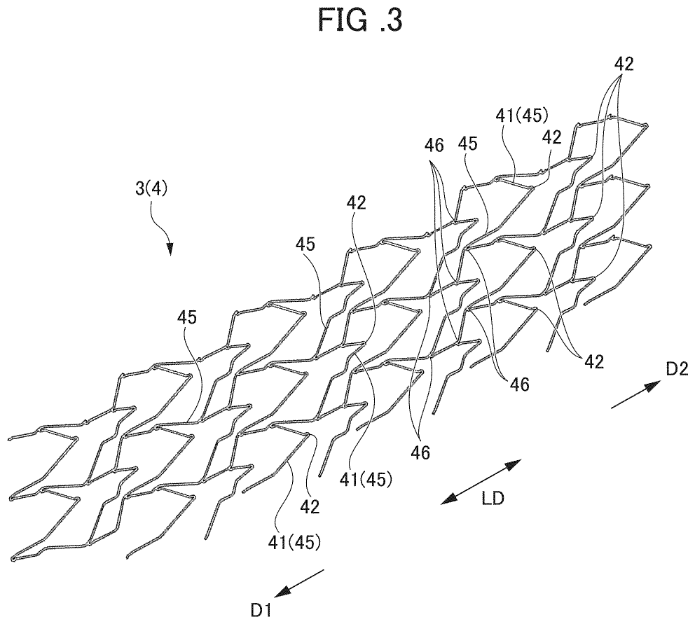

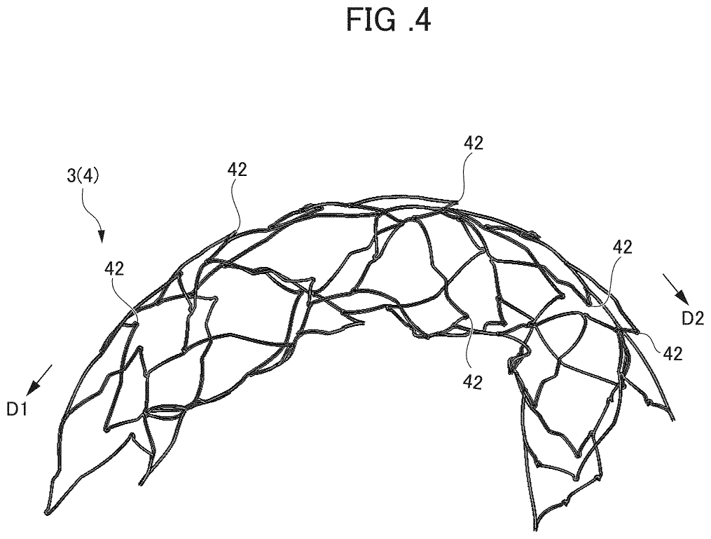

FIG. 1 is a perspective view schematically illustrating the distal stabilizer 1 according to an embodiment of the present invention. FIG. 2 is a perspective view illustrating a state in which a locking stent 3 of the distal stabilizer 1 is imaginarily fitted over a circular column-shaped object. FIG. 3 is a developed view illustrating the locking stent 3 imaginarily developed on a plane. FIG. 4 is a perspective view illustrating the locking stent 3 in a bent state.

The delivery system 10 is a system for delivering a treatment device 7 in a biological lumen V. The treatment device 7 will be described later. As illustrated in FIG. 1, the delivery system 10 includes the distal stabilizer 1, a plurality of catheters including a first catheter 5, a second catheter 6, and a third catheter 8 (not illustrated in FIG. 1).

The distal stabilizer 1 is a device for use to deliver the catheters in the biological lumen V. The distal stabilizer 1 includes a delivery wire 2 as a thread-like delivery member and the locking stent 3 as a locking device stent. The locking stent 3 extends from a distal end 21 of the delivery wire 2, and can lock onto an inner wall V1 of the biological lumen V with its self-expanding force. The biological lumen is not limited, and may be, for example, a blood vessel (artery or vein) of a brain, a coronary vessel (artery or vein), a blood vessel (artery or vein) of an upper or lower limb, or an organ.

The delivery wire 2 is fed toward the distal side D2 when the locking stent 3 is advanced in the biological lumen V, and is pulled back toward the proximal side D1 when the locking stent 3 is retracted in the biological lumen V. The delivery wire 2 may have any diameter as long as the delivery wire 2 has physical properties sufficient for allowing this operation to be performed and fits with the first catheter. The diameter may be, for example, 0.005 inches to 0.018 inches.

The locking stent 3 has a body 4 and an antenna 31. The body 4 has open cells 41 having protruding free ends 42 that protrude only toward the distal side D2. The antenna 31 is a part via which a proximal end 43 of the body 4 converges at the delivery wire 2.

The locking stent 3 may include one locking stent 3 as illustrated in FIG. 1, or two or more locking stents 3 arranged in series. The locking stent 3 preferably has a total effective length of 5 mm or greater. When two or more locking stents are arranged in series, the effective length of each locking stent is not limited, but may be, for example, 1 mm to 5 mm. In the case of the two or more locking stents arranged in series, the delivery wire 2 extends from the antenna of a proximal one of the locking stents to the antenna of the distally next one. Generally, the effective length refers to the length of the body 4.

As illustrated in FIGS. 1 to 4, the body 4 is substantially cylindrical in shape and has a mesh pattern structure. The body 4 has a structure in which a plurality of cells 45 including the plurality of open cells 41 are helically arranged with respect to a long axis direction LD of the locking stent 3. The cell 45 is also referred to as an opening or a compartment, and is a portion surrounded by wire struts that form the mesh pattern of the stent. Among the cells 45, those having the protruding free ends 42 are denoted as the open cells 41. In the present embodiment, all the cells 45 are configured as the open cells. The protruding free end 42 is an end that protrudes in, for example, a substantial V-shape, a substantial U-shape, a substantial Q-shape, or any other similar shapes, and that is not connected to any other strut. It is only necessary for the protruding free end 42 to protrude in a direction approximately toward the distal side D2. The direction is at less than ±90° with respect to the long axis direction LD, preferably at less than ±45° with respect to the long axis direction LD, and more preferably at less than ±22.5° with respect to the long axis direction LD. Since the protruding free ends 42 are not constrained by other struts, its displacement or deformation in a radial direction (a direction perpendicular to the long axis direction LD) is unlikely to be restricted.

The open cells 41 have protruding closed ends 46. The protruding closed end 46 is an end that protrudes in overall view but does not constitute the protruding free end because another strut is connected thereto. The protruding closed end 46 has, for example, a substantially V-shape, a substantially U-shape, or a substantially Q-shape. It is only necessary for the protruding closed end 46 to protrude in a direction approximately toward proximal side D1. The direction is at less than ±90° with respect to the long axis direction LD, preferably at less than ±45° with respect to the long axis direction LD, and more preferably at less than ±22.5° with respect to the long axis direction LD. The protruding closed ends 46 protrude toward the proximal side D1 as described above, but are inhibited from outwardly warping by the struts. Thus, the protruding closed ends 46 are unlikely to become an obstacle to resheathing. In the present embodiment, the protruding free ends 42 each reside between the protruding closed ends 46.

The cells 45 do not all have to be the open cells, but may include the closed cells and the open cells. In the locking stent 3 having the open cells, struts that are bent due to a biological lumen constitute a small area. Therefore, the locking stent 3 with the open cells exerts a less intensive force to straighten the shape of the biological lumen (e.g., a blood vessel) onto which the locking stent 3 locks than a locking stent consisting only of closed cells, and is gentle to biological lumens. For this reason, the locking stent 3 having the open cells is suitable for locking onto a tortuous biological lumen with many twists and turns or a biological lumen where damage can be fatal.

The body 4 is made of a biocompatible material, such as stainless steel, tantalum, platinum, gold, cobalt, nickel, titanium, or an alloy thereof (e.g., a nickel-titanium alloy).

The plurality of open cells 41 are arranged in a circumferential direction. The plurality of open cells 41 arranged in the circumferential direction are inclined with respect to a radial direction. Note that the plurality of open cells 41 arranged in the circumferential direction do not have to be inclined with respect to the radial direction.

The locking stent 3 has a radiopaque portion (not illustrated). The radiopaque portion is a portion having a high radiopacity, and therefore, is highly visible when irradiated with radiation. The radiopaque portion serves as a marker based on which a position of the locking stent 3 is checked. The radiation is, for example, X-rays. Examples of a materials for forming the radiopaque portion include platinum, gold, tantalum, tungsten, iridium, platinum-tungsten, and an alloy material thereof. The examples of the material further include a radiopaque polymer material containing a radiopaque filler or the like added thereto.

Examples of a means for providing the radiopaque portion to the locking stent 3 include the following. A string-shaped radiopaque material is wound around or stretched along the strut. A coil-shaped radiopaque material is wound around a middle portion of the strut or around the protruding end, or is inserted into and engaged with a substantially L-shaped protrusion of the strut (or a pair of L-shaped protrusions facing each other). A layer of a radiopaque material is formed on the strut by, for example, plating or application, whereby a member is formed. The radiopaque portion may be provided over the entire locking stent 3. Alternatively, it may be provided in a portion, such as a distal end 44 of the body 4 or an appropriate location in an intermediate portion of the body in the long axis direction LD.

The catheter includes an elongated cylindrical member that is insertable into a biological lumen V. The catheter may be provided with a radiopaque portion (not illustrated) in a vicinity of a distal end thereof, just like the locking stent 3.

As illustrated in FIG. 1, the first catheter 5 is externally fitted over the delivery wire 2. The first catheter 5 is, for example, a catheter called a microcatheter. The second catheter 6 is externally fitted over the first catheter 5. The first catheter has a diameter that is set according to a target position TP to be described later and an inner diameter and a degree of bend of the biological lumen V constituting a pathway to the target position TP. The inner diameter of the first catheter is not limited, and is preferably 0.017 inches or less, and more preferably 0.0165 inches or less. For the above-described locking stent, the structure and arrangement of the cells and the material and diameter of the struts are designed such that the locking stent fits with the first catheter.

One or more additional catheters (not illustrated) may be used as needed, while being externally fitted over the second catheter 6. In general, using a large number of catheters eventually enables a catheter with a large inner diameter to be inserted and advanced into a biological lumen V.

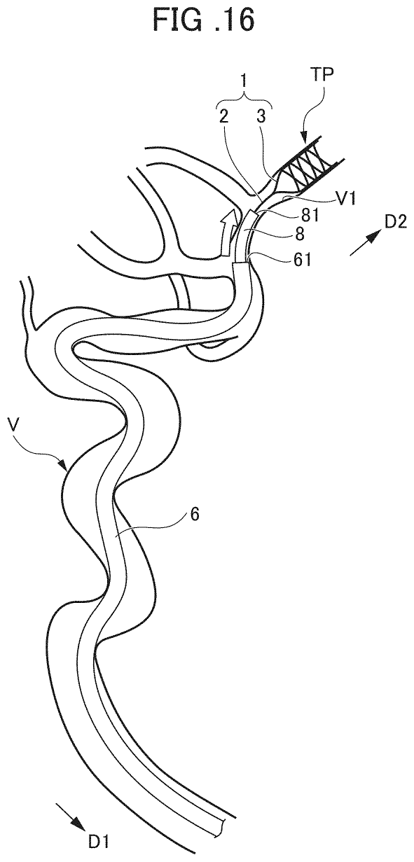

A third catheter (large catheter; denoted by the reference numeral 8 in FIG. 16 to be described later) having an inner diameter larger than the first catheter 5 (small catheter) may be used, as needed. The second catheter 6 having an inner diameter larger than the outer diameter of the third catheter may be used in combination, as needed. The third catheter having the above configuration is easier to manipulate and advance in the biological lumen V toward the distal side than a catheter having an inner diameter of the order of millimeters (typically, the second catheter), and thus, is easy to deliver to a target position adjacent to the distal side. In addition, the third catheter is superior to the first catheter in terms of an ability to deliver a large treatment device or an ability to be used per se as a treatment device. The inner diameter of the third catheter of this aspect is not limited, but may be 0.017 inches or greater (preferably greater than 0.017 inches) and 0.035 inches or less. In the catheter delivery system including the first and third catheters described in this paragraph, the locking stent 3 may include the open cell structure in a portion or the entirety thereof, or may exclude the open cell structure. Specifically, the locking stent of the present embodiment may have a body consisting only of the closed cell structure, and an antenna, as illustrated in Patent Document 1.

Among the plurality of catheters including the second catheter 6, one catheter the inner diameter of which is larger than the first catheter 5 (one catheter the inner diameter of which is sufficient to allow insertion of a treatment device, or to allow itself to be used as a treatment device) is referred to as a purpose catheter PC. The former may be referred to as a guiding catheter while the latter may be referred to as a thrombus suction catheter.

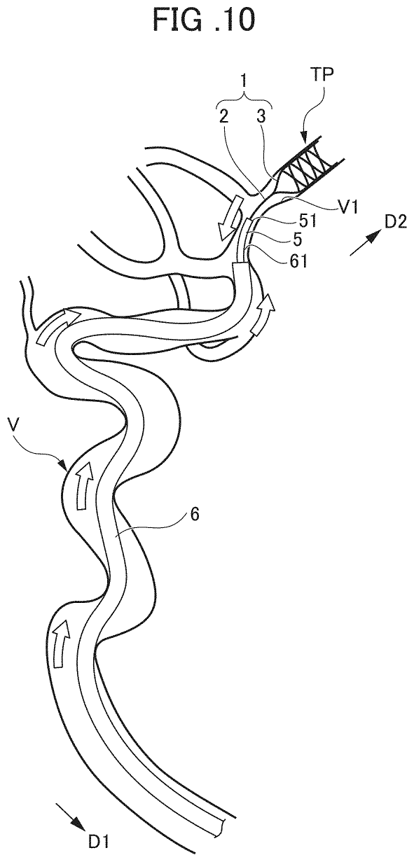

An example case in which the second catheter 6 functions as the purpose catheter PC will be described later with reference to FIGS. 10 to 13. An example case in which the third catheter 8 functions as the purpose catheter PC will be described later with reference to FIGS. 16 to 21.

The distal stabilizer 1 is usable in a catheter delivery operation including: releasing the locking stent 3, which has been loaded in the first catheter 5 while being in a reduced diameter state, from a distal end 51 of the first catheter 5, thereby causing the locking stent 3 to lock onto the inner wall V1; and advancing one catheter (e.g., the second catheter 6) having an inner diameter larger than the first catheter 5 toward the distal side D2 while the locking stent 3 remains locked on the inner wall V1. The distal stabilizer 1 is configured such that after completion of the catheter delivery operation, the locking stent 3 is reduced in diameter to be resheathed into one of the catheters, and the distal stabilizer 1 is removed out of the biological lumen V. In the examples illustrated in the drawings, retracting the delivery wire 2 toward the proximal side D1 in order to remove the distal stabilizer 1 out of the biological lumen V causes the locking stent 3 to decrease in diameter to be resheathed (accommodated) into one of the catheters. The catheter that is advanced toward the distal side D2 after the locking stent 3 is caused to lock onto the inner wall V1 does not have to be only a catheter having an inner diameter larger than the first catheter 5, but may include the first catheter 5 itself or a catheter having an inner diameter equivalent to or smaller than the first catheter 5.

The removal of the distal stabilizer 1 from the biological lumen V can also be performed in the following manner. Prior to retraction of the delivery wire 2 toward the proximal side D1, the catheter is advanced toward the distal side D2 so that the locking stent 3 is reduced in diameter to be resheathed into one of the catheters, and thereafter, the delivery wire 2 is retracted toward the proximal side D1, thereby removing the distal stabilizer 1 out of the biological lumen V.

Next, part of a therapeutic procedure for delivering a desired treatment device 7 to a target position TP using the distal stabilizer 1 and delivery system 10 of the embodiment will be described. The biological lumen V is a blood vessel. In particular, the distal stabilizer 1 and the delivery system 10 of the embodiment are preferably applicable to a case where a blood vessel includes a tortuous blood vessel with many twists and turns, and a case where the target position TP is located in an area with a blood vessel inner diameter of 7 mm or less, specifically, less than 2.5 mm (preferably 2.0 mm or less, or 1.5 mm or less). Specifically, the target position TP may be, for example, one of the M2 and subsequent segments of a middle cerebral artery (MCA) (i.e., M2, M3, M4, etc.), the A1 or A2 segment of an anterior cerebral artery (ACA), one of the P1 and subsequent segments of a posterior cerebral artery (PCA) (i.e., P1, P2, etc.), or an internal carotid artery (ICA). However, the target position TP is not limited to these segments, and may be in an area within a wide range of blood vessel inner diameters from 0.5 mm to 10 mm.

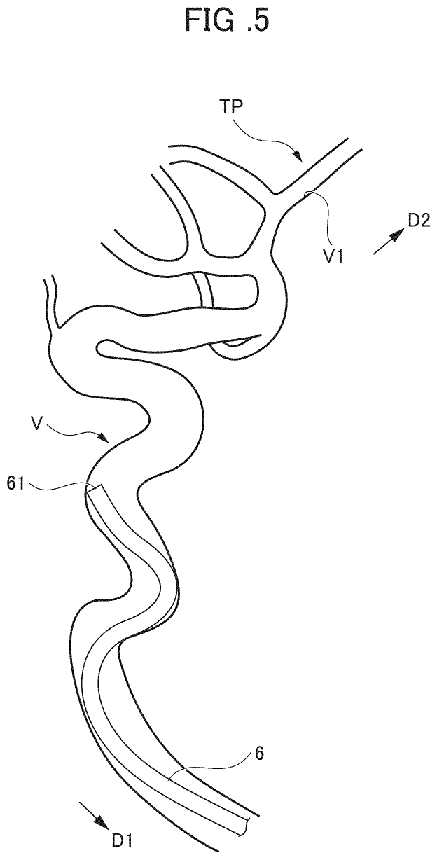

The therapeutic procedure includes various operations in addition to those to be described below. However, a description of such various operations is omitted herein. FIGS. 5 to 12 are schematic diagrams sequentially illustrating a first example of a therapeutic procedure that can be performed using the delivery system 10 including the distal stabilizer 1.

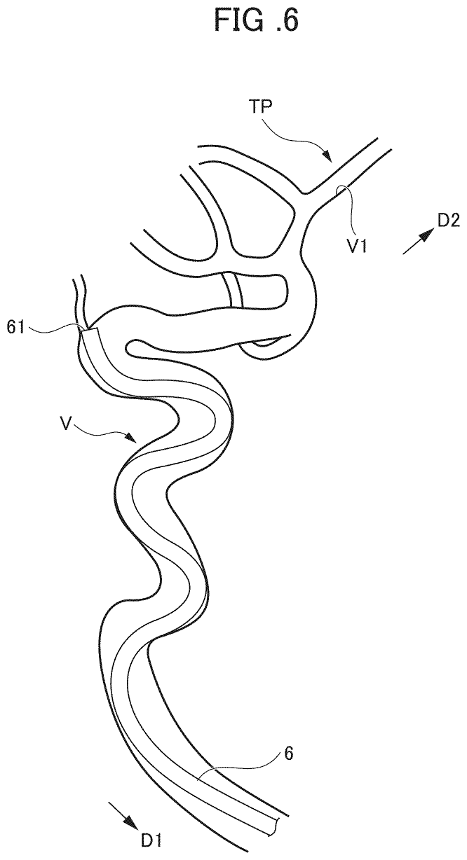

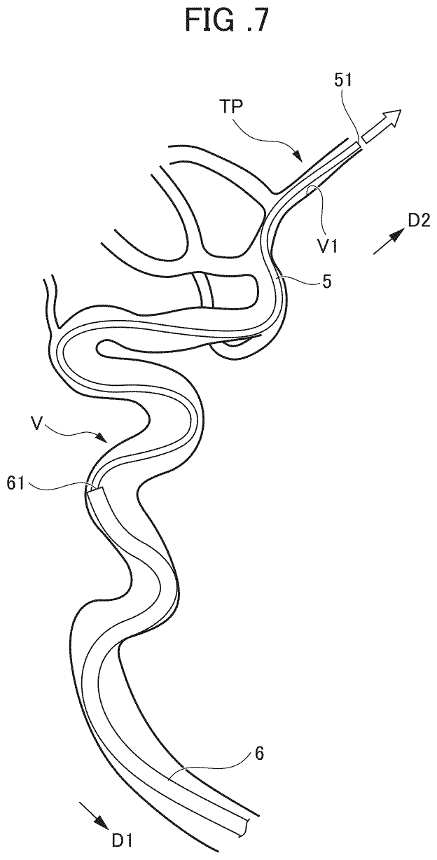

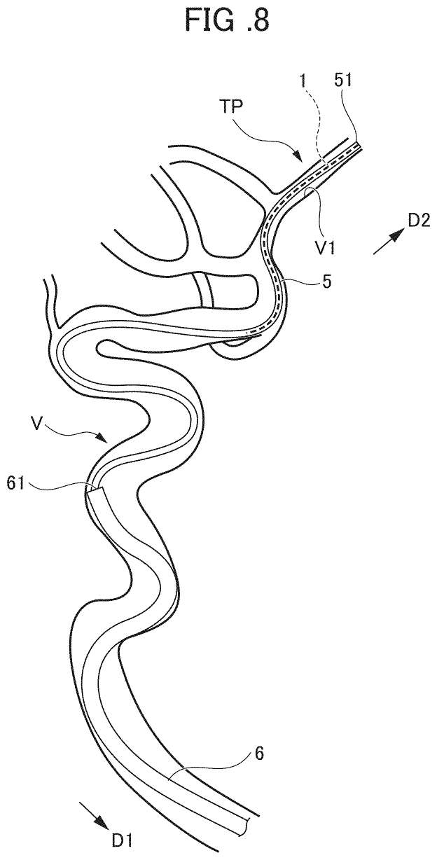

First, the second catheter 6 is placed in a portion of a patient's biological lumen V, the portion being adjacent to the proximal side D1. Typically, as illustrated in FIGS. 5 and 6, the second catheter 6 is caught at its distal end 61 by a bent site or a branch site of the biological lumen V, and therefore, it becomes difficult to advance the second catheter 6 further toward the distal side. As illustrated in FIG. 7, the first catheter 5 is introduced into the biological lumen V by being inserted into the second catheter 6 in the above-described state. The first catheter 5 is then extruded from the distal end 61 of the second catheter 6, and thereafter, the distal end 51 of the first catheter 5 is placed in a vicinity of the target position TP. Subsequently, as illustrated in FIG. 8, the distal stabilizer 1 is inserted into the first catheter 5 to be placed in the vicinity of the target position TP. At this time, the first catheter 5 contains the locking stent 3 of the distal stabilizer 1 loaded therein in the reduced diameter state. Although the distal stabilizer 1 is loaded in, and extends over, the entirety of the first catheter 5, FIG. 8 illustrates, for the sake of convenience, the distal stabilizer 1 only in a distal portion of the first catheter 5, as indicated by the dashed curve.

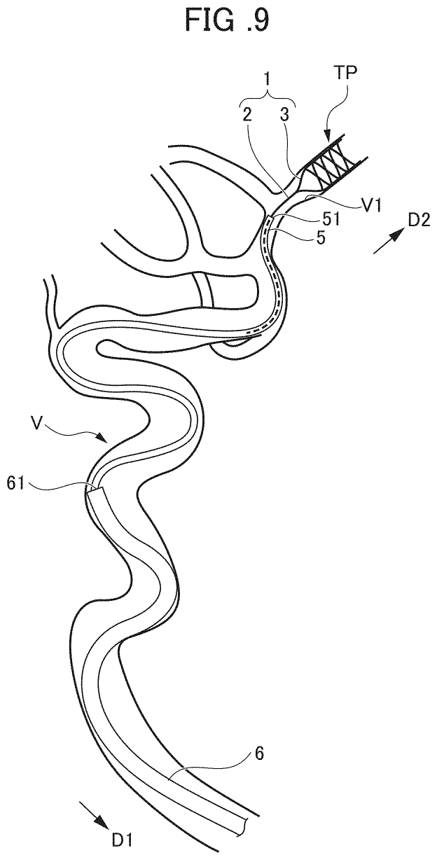

Next, as illustrated in FIG. 9, the locking stent 3 loaded in the first catheter 5 while being in the reduced diameter state is released from the distal end 51 of the first catheter 5. The locking stent 3 is released by way of retracting the first catheter 5 toward the proximal side D1. The locking stent 3 released from the distal end 51 of the first catheter 5 expands by itself with its self-expanding force, presses the inner wall V1 of the biological lumen V outward from the inside of the biological lumen V, and locks onto the inner wall V1. The locking stent 3, which has the open cells 41 with protruding free ends 42, can lock onto the inner wall V1 with a strong locking force.

Next, as illustrated in FIG. 10, the second catheter 6 is externally fitted over the first catheter 5 and advanced in the biological lumen V toward the distal side D2. The second catheter 6 has a large outer diameter and is highly rigid (resilient). Therefore, in the process of advancing, the second catheter 6 repeatedly exerts a force for dragging the first catheter 5 and the delivery wire 2 that are less rigid toward the proximal side. However, the locking stent 3 that firmly locks onto the inner wall V1 can maintain the first catheter 5 and the delivery wire 2 in the placed position against the dragging force exerted by the second catheter 6.

Nevertheless, the second catheter 6, which is highly rigid, may not be able to pass through a sharply bent site or a site with a small inner diameter in the biological lumen V, and may not be able to reach the position where the distal end 51 of the first catheter 5 is present. In this case, the distal end 61 of the second catheter 6 is positioned closer to the proximal side D1 than the distal end 51 of the first catheter 5. The second catheter 6 meanders widely in the biological lumen V due to its high rigidity. Consequently, the second catheter 6 has a long path length. The path length refers to the length of a path to a certain position in the biological lumen V. The path length becomes shortest when the path is straight. However, since the biological lumen V is usually not straight, the path repeatedly bends (meanders). The path length decreases as the path becomes closer to a straight line.

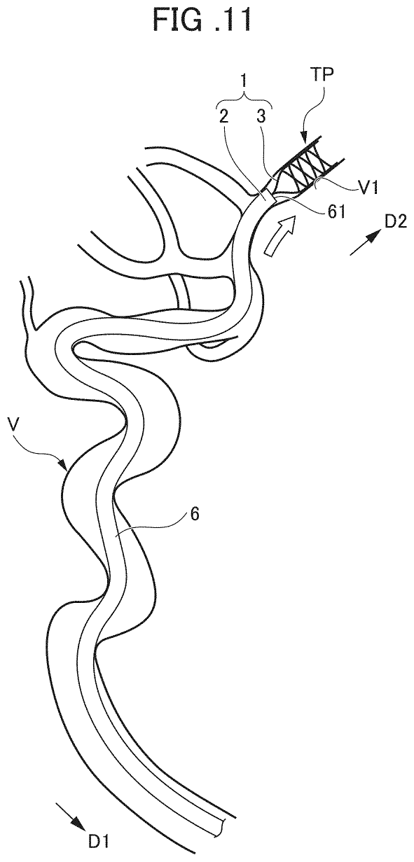

In this case, as illustrated in FIG. 10, the delivery wire 2 can be pulled toward the proximal side D1 while the locking stent 3 remains locked on the inner wall V1. This pulling operation advances the second catheter 6 toward the distal side D2, as illustrated in FIG. 11. Specifically, when the delivery wire 2 is pulled toward the proximal side D1 while the locking stent 3 remains locked on the inner wall V1, the path of the delivery wire 2 becomes close to a straight line and is shortened in the biological lumen V. At this stage, a proximal portion of the second catheter 6 adjacent to the proximal side D1 is retained directly or indirectly, whereby the proximal portion of the second catheter 6 remains at the same position, while the distal end 61 of the second catheter 6 advances in the biological lumen V instead. Further, in this situation where the proximal portion of the second catheter 6 remains at the same position while the distal end 61 of the second catheter 6 advances in the biological lumen V, the path of the second catheter 6 becomes close to a straight line. This operation is particularly useful when the second catheter 6 is intended to pass through a site of the biological lumen V where the passage of the second catheter 6 is difficult, such as a sharply bent site or a site with a small inner diameter.

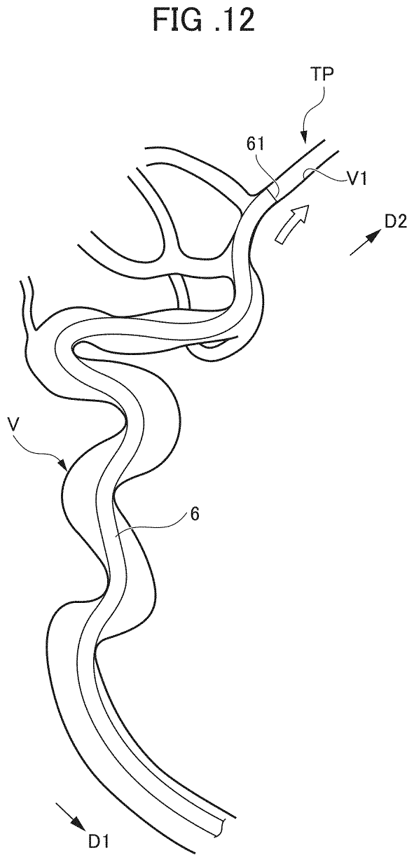

After completion of delivery of the second catheter 6 in this manner, the distal stabilizer 1 is retracted toward the proximal side D1 to be resheathed in the catheter, as illustrated in FIG. 12. Once the distal stabilizer 1 is resheathed in the first catheter 5, the first catheter 5 and the distal stabilizer 1 may be removed from the second catheter 6 on the proximal side D1. Alternatively, following removal of the first catheter 5 from the proximal side D1, the distal stabilizer 1 may be resheathed into the second catheter 6 and removed from the proximal side D1.

Here, the proximal end 43 of the body 4 converges at the delivery wire 2 via the antenna 31 and the protruding free ends 42 of the open cells 41 in the locking stent 3 of the distal stabilizer 1 protrude only toward the distal side D2. For these reasons, in the process of resheathing the locking stent 3 into a catheter, especially the first catheter 5 with a small inner diameter, the locking stent 3 is unlikely to be caught at the distal-side opening of the catheter and is resheathed easily and smoothly.

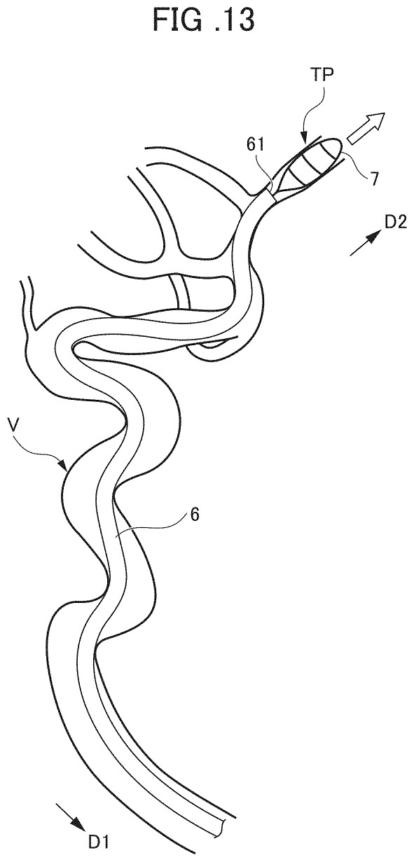

Next, as illustrated in FIG. 13, the second catheter 6 is placed at the target position TP in the biological lumen V so that the treatment device 7 inserted into the second catheter 6 can be easily delivered to the target position TP. Alternatively, the second catheter 6 or the third catheter may be used per se as a treatment device. For example, in the case of using the second catheter 6 as a thrombus suction device (a thrombus suction catheter), the internal flow path of the second catheter 6 is made to have a negative pressure so that a thrombus present at the target position TP is suctioned and removed through the distal end 61. The distal stabilizer 1 may be removed before or after the delivery of the treatment device 7. In the latter case, the second catheter 6 has a doubled or redoubled structure including at least two catheter lumens, one of which allows insertion of the distal stabilizer 1 and the first catheter 5 and another of which allows insertion of the treatment device 7.

The treatment device 7 is, for example, a thrombus suction device, a flow diverter, an aneurysm embolization device, a thrombus removal device (e.g., a stent retriever), an aneurysm treatment stent, an intracranial artery stenosis treatment stent, a balloon catheter, a shunt, or a means for releasing a liquid embolic material (e.g., a catheter with a lumen allowing passage of a liquid embolic material). An example of the thrombus suction device is a thrombus suction catheter. The present invention makes it possible to transcatheterly deliver these treatment devices to the above-mentioned site with a blood vessel inner diameter of 0.5 mm to 10 mm, preferably 7 mm or less, and specifically less than 2.5 mm (preferably 2.0 mm or less, or 1.5 mm or less), and makes it possible to treat diseases or conditions (a vascular occlusion, a vascular aneurysm, etc.) at the site while allowing a small burden on the patient. The treatment device 7 delivered to the target position TP may be used within the biological lumen (in the case of a thrombus suction device, a flow diverter, an aneurysm embolization device, a thrombus removal device, an aneurysm treatment stent, an intracranial artery stenosis treatment stent, a balloon catheter, or a means for releasing a liquid embolic material, etc.), or may be used while projecting outside the biological lumen (in the case of a shunt, etc.). The present invention also encompasses a method including delivering the treatment device 7 to the target position TP in the biological lumen V using the delivery system 10, and administering a treatment at the target position TP by using the treatment device 7 within the biological lumen V or by using the treatment device 7 projecting from the target position TP to the outside of the biological lumen V.

The present embodiment exerts, for example, the following effects.

The distal stabilizer 1 of the present embodiment includes the delivery wire 2 and the locking stent 3 that extends from the distal end 21 of the delivery wire 2 and is capable of locking onto the inner wall V1 of the biological lumen V with its self-expanding force. The locking stent 3 includes the body 4, and the antenna 31 via which the proximal end 43 of the body 4 converges at the delivery wire 2. The body 4 has the open cells 41 with the protruding free end 42 that protrude only toward the distal side D2. The distal stabilizer 1 of the embodiment is usable in the catheter delivery operation including: releasing the locking stent 3, which has been loaded in the first catheter 5 while being in the reduced diameter state, from the distal end 51 of the first catheter 5, thereby causing the locking stent 3 to lock onto the inner wall V1; and advancing the second catheter 6 that is externally fitted over the first catheter 5 toward the distal side D2 while the locking stent 3 remains locked on the inner wall V1. The distal stabilizer 1 is configured such that after completion of the catheter delivery operation, retracting the delivery wire 2 toward the proximal side D1 in order to remove the distal stabilizer 1 out of the biological lumen V causes the locking stent 3 to decrease in diameter to be resheathed into the catheter 5 or 6.

Thus, the distal stabilizer 1 of the present embodiment, which has the open cells 41, exhibits excellent performance in locking onto the inner wall V1 of the biological lumen V. In addition, due to the protruding free ends 42 that protrude only toward the distal side, the distal stabilizer 1 is resheathed into the catheter 5 or 6 considerably reliably and easily when the delivery wire 2 is retracted toward the proximal side D1 in order to remove the distal stabilizer 1 out of the biological lumen V after the completion of the catheter delivery operation. The locking stent 3 can fit with a microcatheter with a small inner diameter (e.g., the first catheter 5 with an inner diameter of 0.017 inches or less, preferably 0.0165 inches or less). These features make it possible to deliver a catheter, and accordingly, a treatment device to a peripheral blood vessel where such delivery has traditionally been considered to be impossible. The inner diameter of the first catheter 5 with which the locking stent 3 fits is not limited, and may exceed 0.017 inches. The distal stabilizer 1 of the present embodiment, which has open cells 41 and is gentle to biological lumens, is also suitable for locking in a tortuous site with many twists and turns or a site where damage can be fatal, such as a peripheral blood vessel.

When the distal stabilizer 1 of the present embodiment is used, the second catheter 6 is advanced by way of pulling the delivery wire 2 toward the proximal side D1 while the locking stent 3 remains locked on the inner wall V1. According to the distal stabilizer 1 of the present embodiment, the pulling operation causes the delivery wire 2 and the catheters 5 and 6 provided over the delivery wire 2 to be placed so as to draw the shortest paths, resulting in that the catheters 5 and 6 are allowed to move toward the distal side D2 by a distance corresponding to a difference from the shortest path.

According to the distal stabilizer 1 of the present embodiment, the locking stent 3 is released by way of retracting the first catheter 5 toward the proximal side D1. Therefore, the distal stabilizer 1 of the present embodiment allows a practitioner to release the locking stent 3 from the first catheter 5 while inhibiting the protruding free ends 42 protruding toward the distal side D2 from being stabbed into the inner wall V1 of the biological lumen V.

In the distal stabilizer 1 of the present embodiment, the body 4 has a structure in which the plurality of cells 45 are arranged helically with respect to the long axis direction LD of the locking stent 3. Therefore, according to the present embodiment, the distal stabilizer 1 easily follows the bends of the biological lumen V. The distal stabilizer 1 can exert a greater locking force when it locks in a sharply bent site than when it locks in a gently bent site. Many of such sharply bent sites are located near the peripheral side of a biological lumen V. The distal stabilizer 1 of the present embodiment fits with a microcatheter having a small inner diameter, and thus, provides an advantage that it can be easily delivered to such a sharply bent site.

The locking stent 3 of the distal stabilizer 1 of the present embodiment has a total effective length of 5 mm or greater. Therefore, according to the distal stabilizer 1 of the present embodiment, the large effective length of the locking stent 3 makes it easy for the locking stent 3 to exert the locking force onto the inner wall V1 of the biological lumen V. Although it would become more difficult to resheathe the locking stent 3 into a catheter as the effective length increases, the structure of the locking stent 3 of the present embodiment can avoid this disadvantage. From this viewpoint, the total effective length of the locking stent 3 may be 7.5 mm or greater, 10 mm or greater, or 12.5 mm or greater. On the other hand, if the total effective length is too long, the locking stent 3 becomes poorly slidable in the catheter, and its operability is deteriorated. For this reason, the total effective length of the locking stent 3 is preferably 45 mm or less, more preferably 40 mm or less, 30 mm or less, or 25 mm or less.

In the following, embodiments that differ from the first embodiment will be described. A description of the same aspects as those of the first embodiment will be omitted partly.

In the distal stabilizer 1 of the embodiment, the locking stent 3 has the radiopaque portion. Therefore, according to the distal stabilizer 1 of the embodiment, positioning of the locking stent 3 is easily performed.

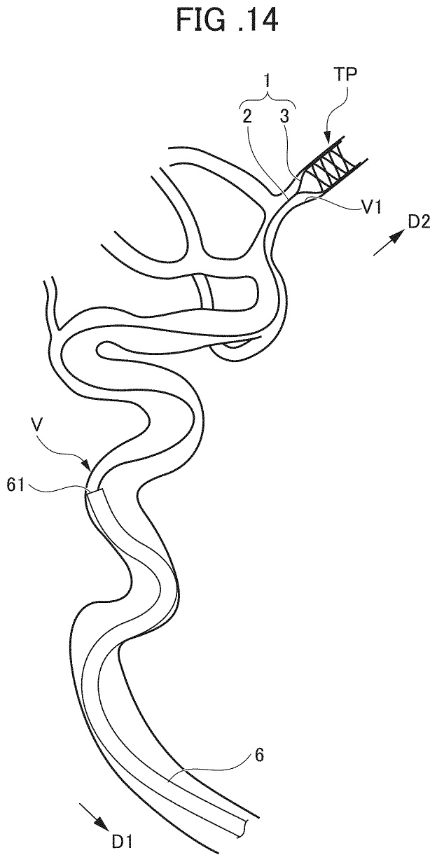

Next, a second embodiment of the present invention will be described with reference to FIGS. 14 to 18. FIG. 14 is a diagram schematically illustrating another embodiment of the process subsequent to the process illustrated in FIG. 9. As illustrated in FIG. 14, after the locking stent 3 is caused to lock onto the inner wall V1, the first catheter 5 (small catheter) is retracted toward the proximal side D1, and then, removed.

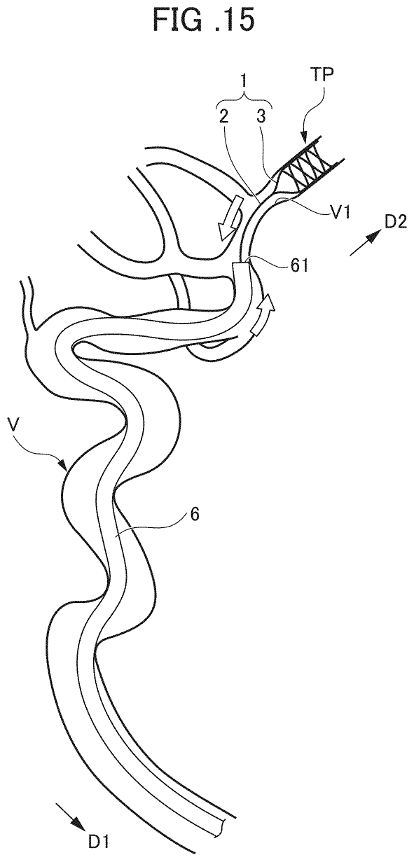

Next, as illustrated in FIG. 15, the second catheter 6 (large catheter) externally fitted over the delivery wire 2 is advanced in the biological lumen V toward the distal side D2. Since the locking stent 3 remains firmly locked on the inner wall V1, the delivery wire 2 can be maintained in the placed position against a dragging force exerted by the second catheter 6.

Next, as illustrated in FIG. 16, a third catheter 8 (large catheter) is externally fitter over the delivery wire 2, and is inserted into the second catheter 6 where the first catheter 5 is now absent. While maintaining this state, the third catheter 8 is extruded from the distal end 61 to be delivered to the target position TP. At this stage, the third catheter 8 can be easily delivered to the distal end 61 since it is supported on the second catheter 6 that is highly rigid. Further, after being extruded from the distal end 61, the third catheter 8 can be easily delivered to the target position TP since it is supported on the delivery wire 2 that is firmly positioned due to the strong locking force of the locking stent 3. The outer diameter of the third catheter 8 is smaller than the inner and outer diameters of the second catheter 6. Therefore, in a case where the biological lumen has a sharply bent site or site with a small diameter at the target position TP or in the route to the target position TP, the third catheter 8 can be more easily delivered to the target position TP than the second catheter 6.

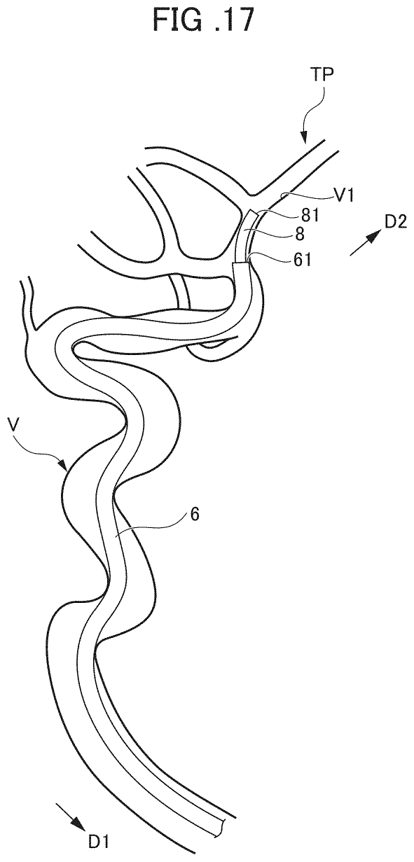

Thereafter, as illustrated in FIG. 17, the delivery wire 2 is retracted toward the proximal side D1, so that the locking stent 3 decreases in diameter to be resheathed into the third catheter 8, and the distal stabilizer 1 is removed out of the biological lumen.

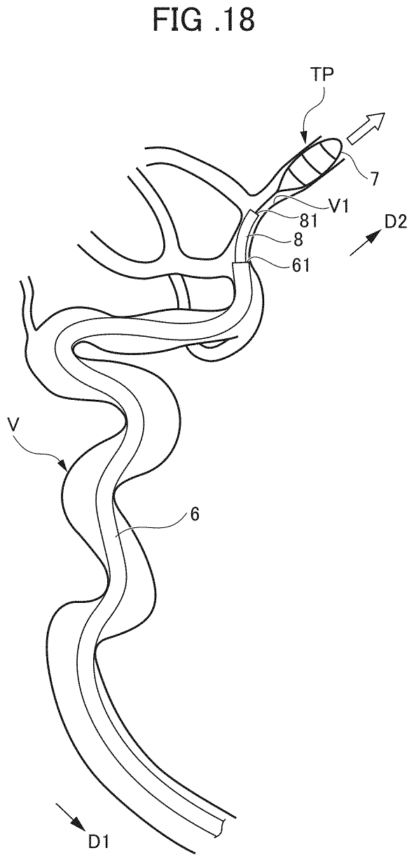

Next, as illustrated in FIG. 18, the treatment device 7 is inserted into the third catheter 8, and then, is extruded from a distal end 81 of the third catheter 8, thereby delivering the treatment device 7 to the target position TP. Since the inner diameter of the third catheter 8 is larger than that of the first catheter 5, a high degree of freedom is provided with respect to the size of the treatment device 7 that can be delivered. This feature makes it possible to administer a treatment using a treatment device optimized for a symptom of interest, such as a flow diverter or an aneurysm embolization device. The third catheter may be used per se as a treatment device.

The distal stabilizer 1 may be removed before or after the delivery of the treatment device 7. In the latter case, the third catheter has a doubled or redoubled structure including at least two catheter lumens, one of which allows insertion of the distal stabilizer 1 and another of which allows insertion of the treatment device 7.

A third embodiment of the present invention differs from the second embodiment in that the third embodiment excludes the operation of advancing the second catheter to the vicinity of the target position TP after removal of the first catheter.

First, the distal end 51 of the first catheter 5 (small catheter) is placed in the vicinity of the target position TP (FIG. 7).

Subsequently, the distal stabilizer 1 is inserted into the first catheter 5 to be placed in the vicinity of the target position TP (FIG. 8).

Next, the locking stent 3 loaded in the first catheter 5 while being in the reduced diameter state is released from the distal end 51 of the first catheter 5. The locking stent 3 is released by way of retracting the first catheter 5 toward the proximal side D1 (FIG. 9).

After the locking stent 3 is caused to lock onto the inner wall V1, the first catheter 5 is retracted toward the proximal side D1 to be removed (FIG. 14).

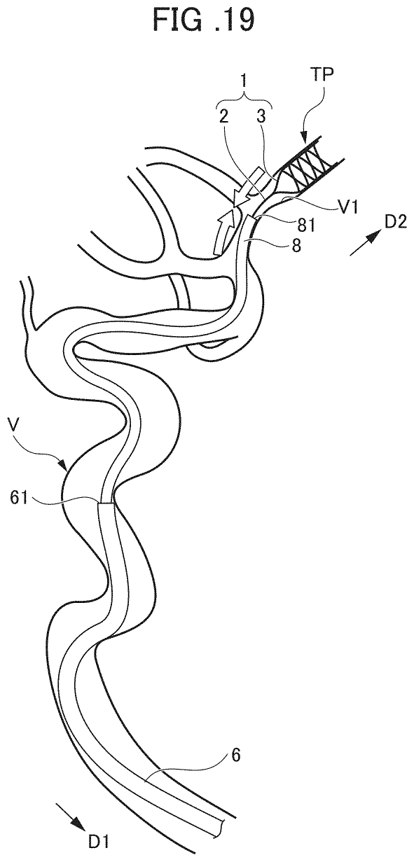

Next, as illustrated in FIG. 19, the third catheter 8 (large catheter) is externally fitted over the delivery wire 2, and is delivered to the target position TP. At this time, the third catheter 8 can be easily delivered to the target position TP since it is supported on the delivery wire 2 that is firmly positioned due to a strong locking force of the locking stent 3.

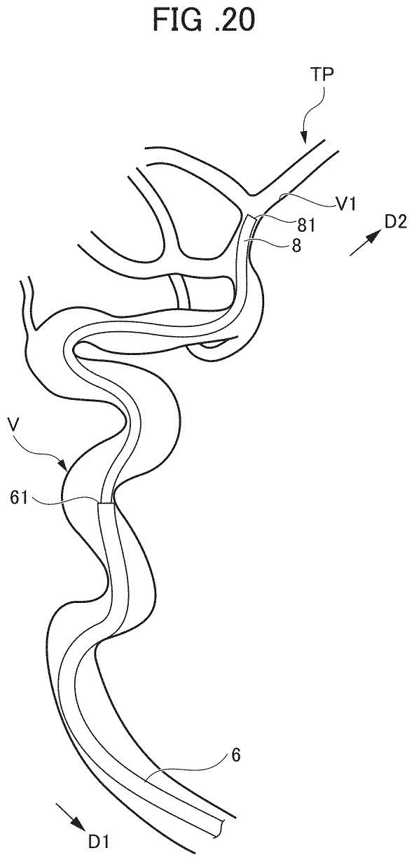

At this stage, the delivery wire 2 can be pulled toward the proximal side D1 while the locking stent 3 remains locked on the inner wall V1. This pulling operation advances the third catheter 8 toward the distal side D2. This operation is particularly useful when the third catheter 8 is intended to pass through a site of the biological lumen V where the passage of the third catheter 8 is difficult, such as a sharply bent site or a site with a small inner diameter. Thereafter, as illustrated in FIG. 20, the delivery wire 2 is retracted toward the proximal side D1, so that the locking stent 3 decreases in diameter to be resheathed into the third catheter 8, and then, the distal stabilizer 1 is removed out of the biological lumen.

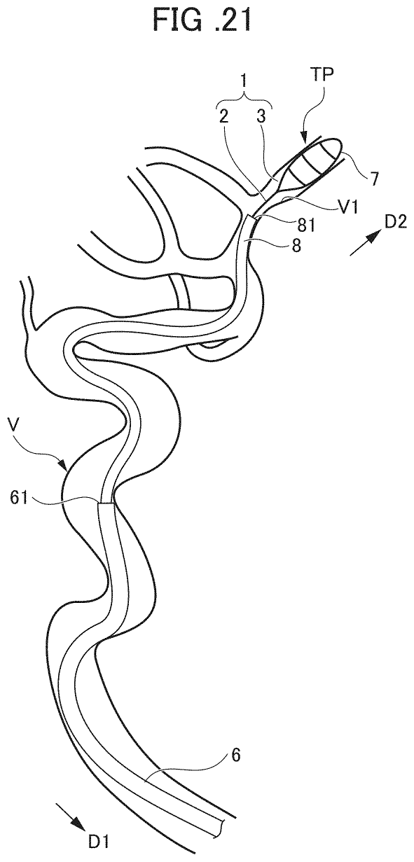

Subsequently, the treatment device 7 is inserted into the third catheter 8, and then, is extruded from the distal end 81, thereby delivering the treatment device 7 to the target position TP (FIG. 21). The third catheter may be used per se as a treatment device.

The distal stabilizer 1 may be removed before or after the delivery of the treatment device 7. In the latter case, the second catheter has a doubled or redoubled structure including at least two catheter lumens, one of which allows insertion of the distal stabilizer 1 and another of which allows insertion of the treatment device 7.

The catheter into which the locking stent 3 is resheathed is not limited to the third catheter 8, but may be a fourth catheter newly inserted into the biological lumen. The fourth catheter may be larger than, or equivalent to or smaller than the inner diameter of the first catheter. In the latter case, the fourth catheter can be the first catheter itself.

A fourth embodiment of the present invention is directed to a system that differs from the second and third embodiments in that a locking stent of the fourth embodiment has a filter function. The description of the foregoing locking stent 3 is appropriately applied to or incorporated into the following description of the locking stent 3A of the fourth embodiment.

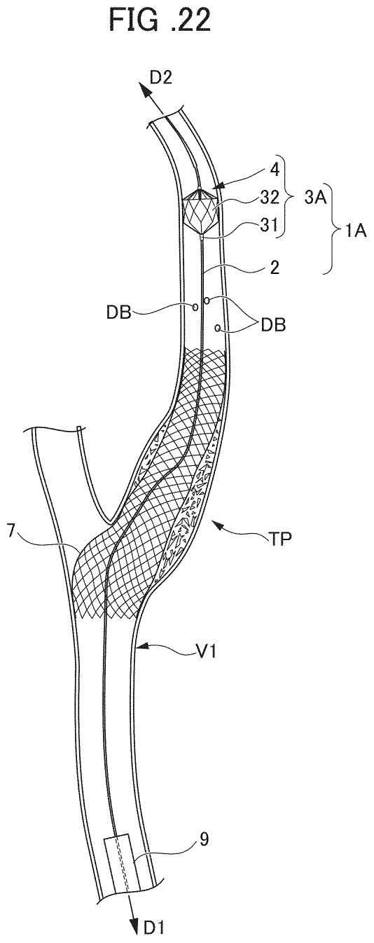

As illustrated in FIG. 22, the locking stent 3A further has a filter 32 that fills a gap over the entire circumferential direction of a body 4. The filter 32 includes a water-impermeable hydrophobic material (e.g., fluoroplastics such as PTFE) and has a large number of pores of a size (e.g., a pore diameter of 0 μm to 100 μm) that allows neither solids (e.g., debris DB to be described later) nor blood to pass, or a size (e.g., a pore diameter of 30 μm to 100 μm) that blocks solids but allows blood to pass. A pore diameter of 0 μm means the absence of the pores.

While the thus-configured locking stent 3A is locked at a position closer to the distal side D2 than a target position TP, a treatment device 7 (a stenosis treatment device such as a balloon or an indwelling stent) is transcatheterly delivered to the target position TP where a stenosis resides. The delivery process is not limited, but may be the same as the procedure of the third embodiment illustrated in FIG. 19 and the preceding figures.

Thereafter, the treatment device 7 is used at the target position TP while the locking stent 3A remains locked without being removed. At this time, the debris DB produced due to crushing of part of a biological material forming the stenosis may flow in the bloodstream toward the distal side D2 and may cause an adverse event. According to the present embodiment, the locking stent 3A, which has a filter function (the filter 32) and is placed closer to the distal side D2 than the target position TP, can filter out the debris DB without stopping the blood flow.

Thereafter, the delivery wire 2 is retracted toward the proximal side D1 so that the locking stent 3A decreases in diameter to be resheathed into the catheter. The distal stabilizer 1A can be removed out of the biological lumen together with the filtered debris DB. Alternatively, it is possible to make the locking stent 3A indwell in the biological lumen V by separating the locking stent 3A from the delivery wire 2. The locking stent 3A can be separated by any process. It can be separated by known means (e.g., breaking a coupling structure with heating by electrical conduction or a physical force or the like).

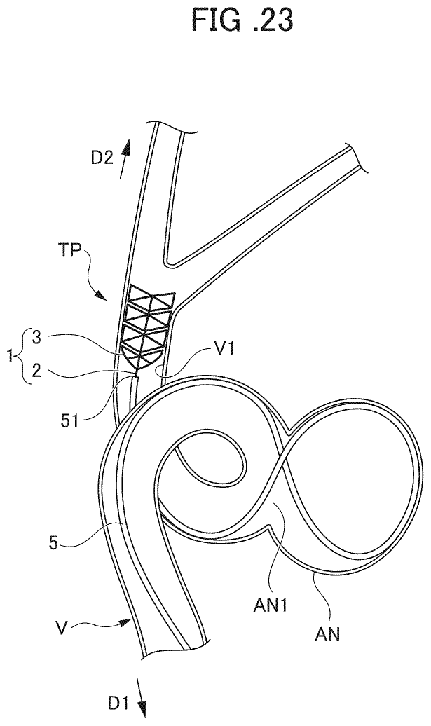

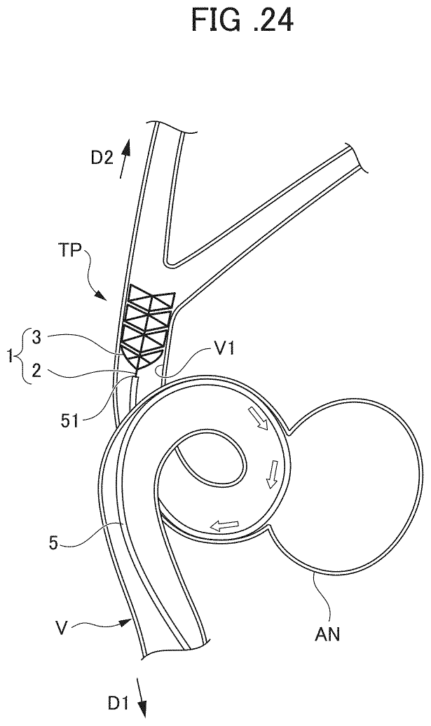

Next, a fifth embodiment of the present invention will be described with reference to FIGS. 23 and 24. In the following, aspects that differ from those of the first to fourth embodiments will be mainly described, and a description of shared aspects will be omitted partly. A distal stabilizer 1 of the fifth embodiment is configured such that a locking stent 3 is loaded in a small catheter (a first catheter 5 in the present embodiment) while being in a reduced diameter state, and the thus-loaded locking stent 3 is released from a distal end of the small catheter at a site distal to an aneurysm AN and is caused to lock onto an inner wall V1. A purpose catheter including a large catheter and externally fitted over a delivery wire 2 is advanced toward the distal side D2 and placed at a target position TP in a biological lumen V. In this way, the distal stabilizer 1 can be used to enable delivery of a treatment device as the purpose catheter itself or a treatment device inserted into the purpose catheter to the target position TP. The target position TP is the aneurysm AN or a position distal to the aneurysm AN.

As illustrated in FIG. 23, the locking stent 3 is caused to lock onto the inner wall V1 of the biological lumen V at a location closer to the distal side D2 than the aneurysm AN. If the position of the aneurysm AN in a blood vessel (a typical but non-limiting example of which is a very tortuous cerebral blood vessel) as the biological lumen V is closer to the proximal side D1 than the site where the locking stent locks, the delivery wire 2 and the first catheter 5 may sink into the aneurysm AN and become unable to advance in an intended direction, resulting in that performance in delivering the catheters (e.g., the second catheter and the third catheter) having an inner diameter larger than the first catheter 5 can be deteriorated significantly. The system of the present embodiment is also suitable for use in cases where the tortuosity of the biological lumen V is gentle before and after an aneurysm AN.

In the above case, as illustrated in FIG. 24, pulling the delivery wire 2 toward the proximal side D1 while the locking stent 3 remains locked on the inner wall V1 shortens the path of the delivery wire 2 in the biological lumen V and allows the delivery wire 2 and the first catheter 5 to return to the biological lumen V from within the aneurysm AN. This operation allows the ability to advance in the intended direction to be restored and makes it possible to easily place the catheters (e.g., the second and third catheters) having an inner diameter larger than the first catheter 5 at the aneurysm AN or the target position TP located closer to the distal side D2 than the aneurysm AN.

If the target position TP is an aneurysm AN, the delivery wire 2 and the first catheter 5 can be placed along an opening AN1 (see FIG. 23) of the aneurysm AN by appropriately performing the above-mentioned pulling operation and, if necessary, a subsequent advancing operation. In this state, a purpose catheter (e.g., the second or third catheter) having an inner diameter larger than the first catheter 5 is placed along the opening AN1 of the aneurysm AN, and thereafter, a treatment device inserted into the purpose catheter is delivered to the aneurysm AN.

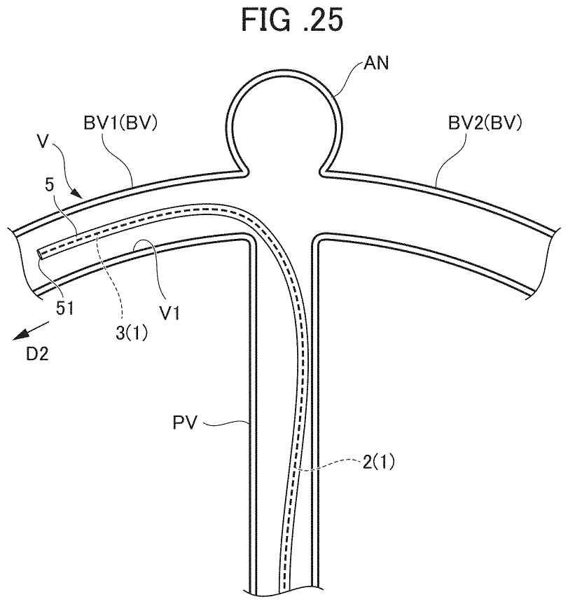

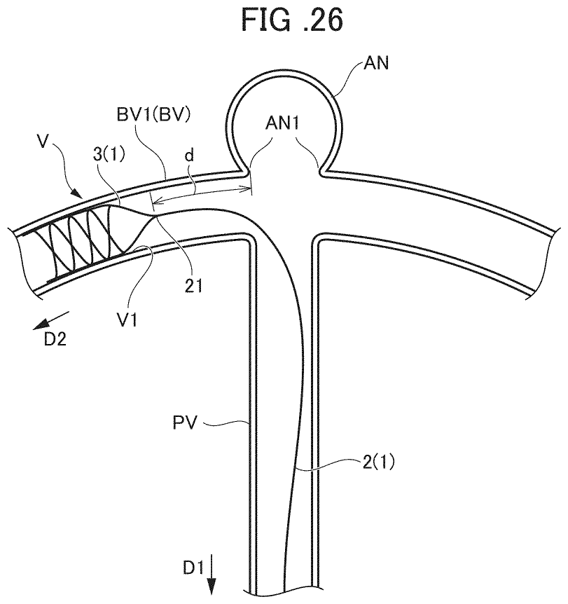

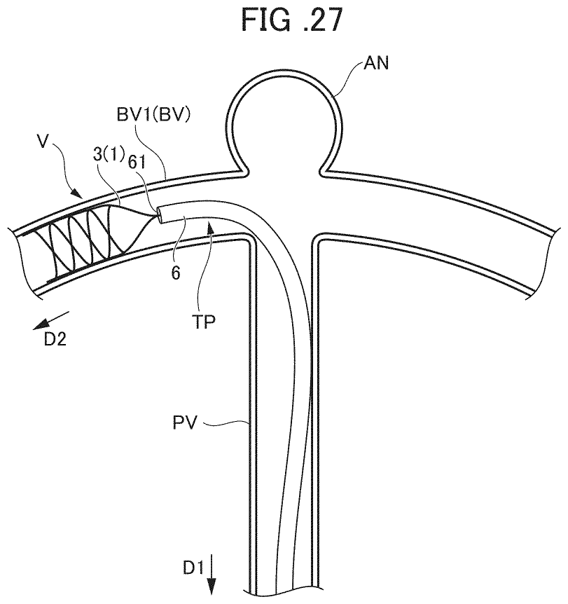

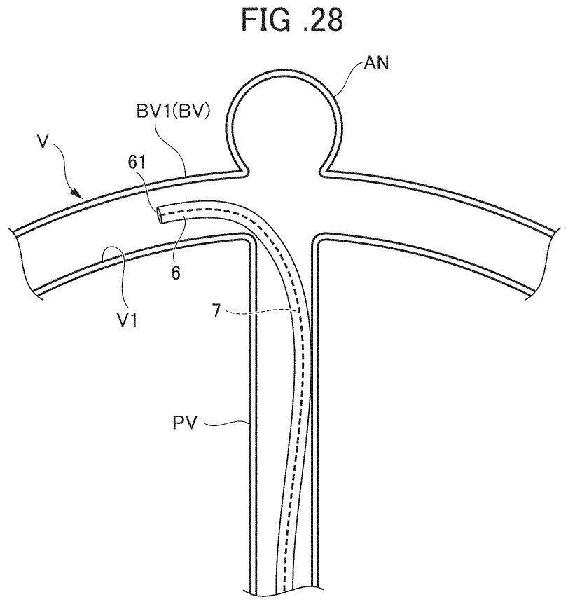

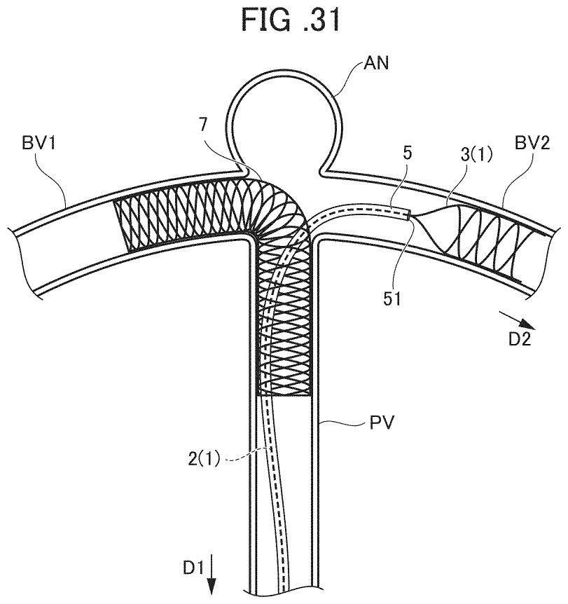

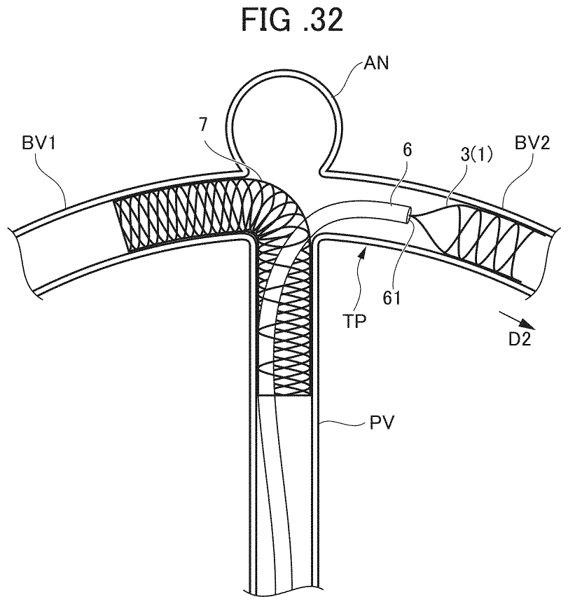

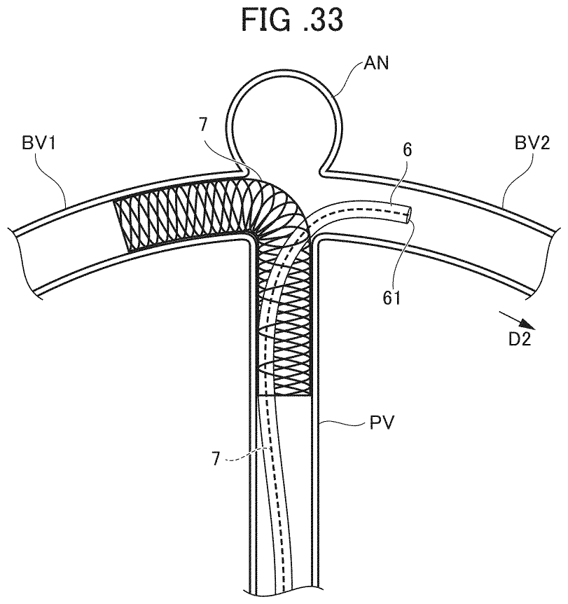

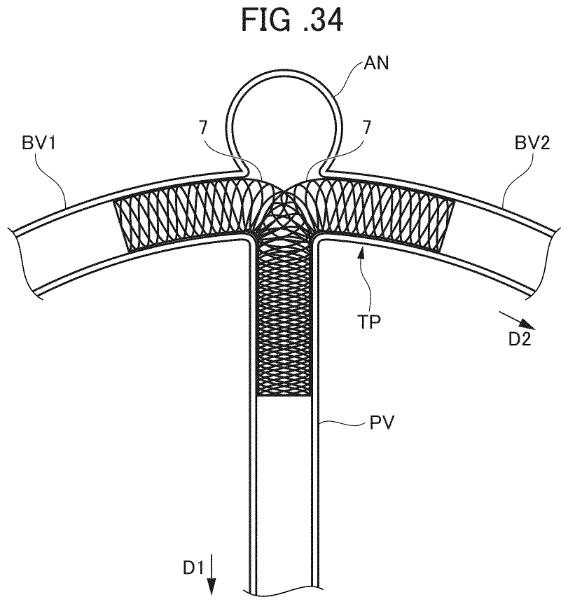

Next, a sixth embodiment of the present invention will be described with reference to FIGS. 25 to 35. In the following, aspects that differ from those of the first to fifth embodiments will be mainly described, and a description of share aspects will be omitted partly. A distal stabilizer 1 of the sixth embodiment is configured such that a locking stent 3 (FIGS. 25 and 26) is loaded in a small catheter (a first catheter 5 in the present embodiment) while being in a reduced diameter state, and the thus-loaded locking stent 3 is released from a distal end of the small catheter at a site closer to the distal side D2 than a bifurcation aneurysm AN and is caused to lock onto an inner wall V1 of a biological lumen V (FIG. 26). A purpose catheter including a large catheter 6 and externally fitted over a delivery wire 2 is advanced toward the distal side D2 (FIG. 27) to be placed at a target position TP in the biological lumen V. In this way, the distal stabilizer 1 can be used to enable delivery of a treatment device as the purpose catheter itself or a treatment device as a flow diverter 7 inserted in the purpose catheter to the target position TP (FIG. 28). The target position TP is an area extending from a location closer to the proximal side D1 than the bifurcation aneurysm AN to a bifurcation vessel BV. The sixth embodiment will be described in detail.

As illustrated in FIG. 25, the distal end 51 of the first catheter 5 is passed through a proximal vessel PV and is placed at a site closer to the distal side D2 than the bifurcation aneurysm AN, specifically, in one bifurcation vessel BV1 of the bifurcation vessels BV between which the bifurcation aneurysm AN resides. Thereafter, the locking stent 3, which has been loaded in the first catheter 5 while being in a reduced diameter state, is advanced to a vicinity of the distal end 51 (FIG. 25).

Next, the first catheter 5 is retracted toward the proximal side D1, so that the locking stent 3 is released from the distal end 51 of the first catheter 5 and caused to lock onto an inner wall V1 of the bifurcation vessel BV1 (FIG. 26). Then, the second catheter 6 (large catheter) externally fitted on the delivery wire 2 in this state is advanced and placed at the target position TP (FIG. 27). In many cases, the distal end 21 of the delivery wire 2 is placed at a short distance d (typically within 2 cm) from an opening AN1 of the bifurcation aneurysm AN at this stage, while the proximal vessel PV and the bifurcation vessel BV1 that are the biological lumens in the vicinity of the bifurcation aneurysm AN are considerably tortuous. For this reason, a situation can be caused in which the delivery wire 2 does not remain in the bifurcation vessel BV1 and enters into the bifurcation aneurysm AN, due to being unable to withstand a dragging force exerted by the second catheter 6, which can be large in diameter and rigid. However, according to the present embodiment, since the locking stent 3 firmly locks on the inner wall V1, the delivery wire 2 can remain in the bifurcation vessel BV1 against the dragging force of the second catheter 6. Therefore, in the present embodiment, a catheter with a desired large diameter (e.g., an outer diameter of 1.0 mm or greater, preferably 1.5 mm or greater, or 2.0 mm or greater) can be suitably used as the second catheter 6.

FIGS. 26 and 27 illustrate that after the removal of the first catheter 5 toward the proximal side D1, the second catheter 6 is advanced along the exposed delivery wire 2. However, this is a non-limiting example, as apparent from the foregoing embodiments. As described in the foregoing embodiments, pulling the delivery wire 2 toward the proximal side D1 while the locking stent 3 remains locked on the inner wall V1 shortens the path of the delivery wire 2 in the biological lumen V and allows the delivery wire 2 and the first catheter 5 to return to the biological lumen V from within the bifurcation aneurysm AN.

After completion of the delivery of the second catheter 6 in the manner described above, the distal stabilizer 1 is retracted toward the proximal side D1 to be resheathed in the second catheter 6, and removed. A treatment device 7 is then inserted into the second catheter 6 (FIG. 28), and then, is extruded from the distal end 61 to be delivered to the target position TP.

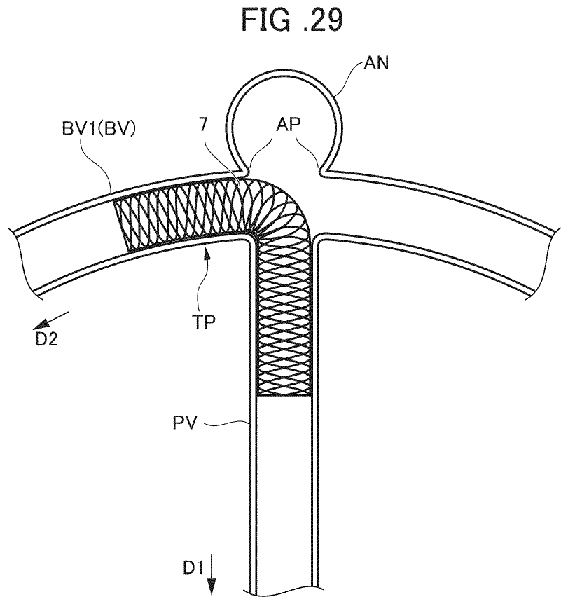

FIG. 29 illustrates a state in which a flow diverter 7 as the treatment device indwells at the target position TP, while the second catheter 6 has been removed. The treatment device 7 that is placed to extend from a proximal site (proximal vessel PV) of the aneurysm AN to the bifurcation vessel BV1 at least partially closes the opening of the bifurcation aneurysm AN. Therefore, a treatment can be administered by filling the bifurcation aneurysm AN with a treatment device, such as an aneurysm embolization device, while the treatment device is supported on the flow diverter 7.

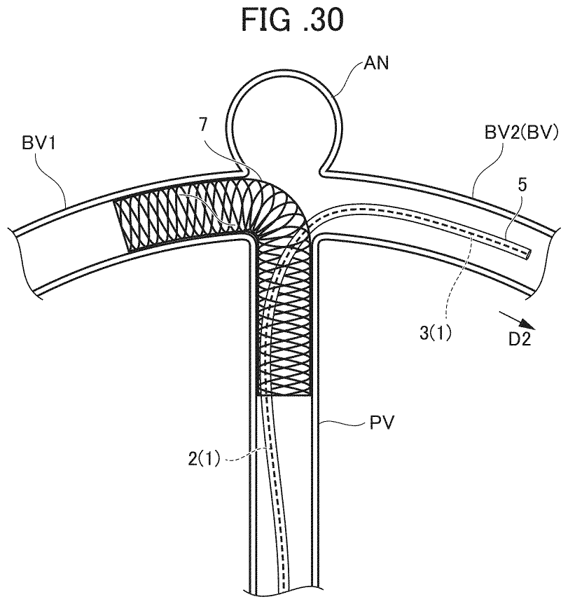

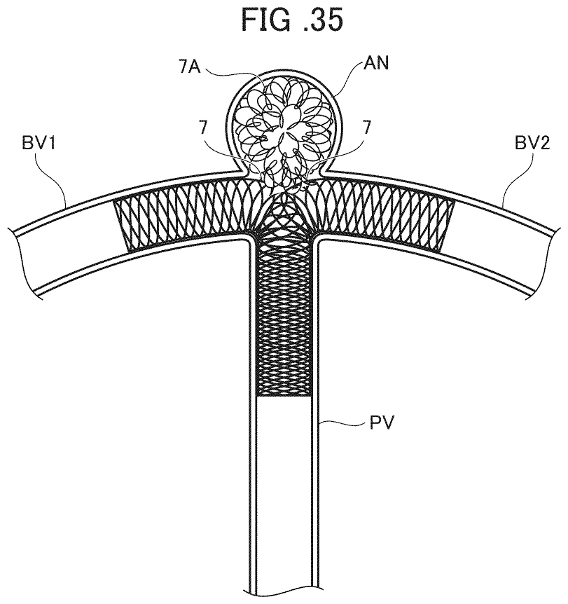

FIGS. 30 to 35 illustrate a procedure for treating a bifurcation aneurysm using two or more delivery systems in combination. In a state as illustrated in FIG. 29, another first catheter 5 is advanced to the other bifurcation vessel BV2 where the treatment device 7 does not indwell, and the same operation as in FIGS. 25 to 29 is performed in the bifurcation vessel BV2 (FIGS. 30 to 34). The other first catheter 5, another second catheter 6, and another treatment device 7 are advanced into the bifurcation vessel BV2 through a gap of the indwelling treatment device 7. As a result, the opening of the bifurcation aneurysm AN is nearly completely closed, and a treatment can be administered more easily by filling the bifurcation aneurysm AN with a treatment device 7A, such as an aneurysm embolization device, while treatment device 7A is supported on the plurality of flow diverters 7, 7 (FIG. 35).

In the first to fifth embodiments, the first to fourth catheters may each independently be of a type including a catheter lumen through which a wire is inserted and which extends from the distal end to the proximal end (the so-called over-the-wire type), or of a type including a catheter lumen which extends from the distal end to an intermediate point before the proximal end, and then, extends to a side surface from the intermediate point (the so-called rapid-exchange type).

From the viewpoint of facilitation and simplification of the delivery operation, if the third catheter 8 is of the rapid-exchange type, at least the first catheter 5 and the second catheter 6 are preferably but not necessarily of the rapid-exchange type. If the third catheter 8 is of the over-the-wire type, the first catheter 5 and the second catheter 6 may each independently be of the over-the-wire type or the rapid-exchange type.

In the delivery system for catheters and treatment devices according to the second to fifth embodiments, the locking stent 3 may have a body consisting only of the closed cell structure and an antenna.

In the foregoing, embodiments of the present invention have been described. However, the above-described embodiments are not intended to limit the present invention, and diverse variations and modifications are possible and encompassed in the technical scope of the present invention. The effects described in the above embodiments are most preferred ones of the effects exerted by the present invention. The effects of the present invention are not limited to those described in the embodiments.

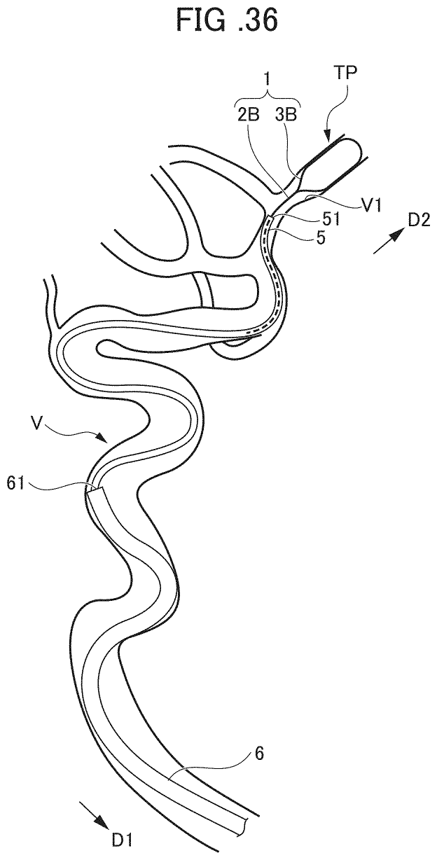

In the present invention, “the locking stent 3 that extends from a distal end of the delivery wire 2 and is capable of locking onto an inner wall of the biological lumen with a self-expanding force” may be replaced with “a locking device that extends from a distal end of a thread-like delivery member and is capable of locking onto an inner wall of the biological lumen with an expanding force”. The locking device is, for example, a balloon. The thread-like delivery member is, for example, a hollow resin tube. As illustrated in FIG. 36, a balloon 3B is an example of “a locking device that extends from a distal end of a hollow resin tube 2B and is capable of locking onto an inner wall of the biological lumen with an expanding force”. The hollow portion of the resin tube 2B allows a liquid (e.g., a mixture of physiological saline and a contrast medium) or a gas to pass therethrough to feed it into the balloon 3B.

The balloon 3B that has been loaded in a first catheter 5 while being in a reduced diameter state is released from a distal end 51 of the first catheter 5. The release of the balloon 3B is performed by way of retracting the first catheter 5 toward the proximal side D1 or advancing the balloon 3B toward the distal side D2. The balloon 3B released from the distal end 51 of the first catheter 5 is inflated and expanded by a liquid fed through the resin tube 2B into the balloon 3B. As a result, the balloon 3B generates an expanding force, presses the inner wall V1 of the biological lumen V outward from the inside of the biological lumen V, and locks onto the inner wall V1. The balloon 3B in an expanded state can lock onto the inner wall V1 with a locking force associated with the expanding force.

On the other hand, the balloon 3B in the expanded state is deflated and contracted by way of suctioning and removing the fed liquid from inside the balloon 3B through the resin tube 2B. As a result, the balloon 3B is unlocked from the inner wall V1. The unlocked balloon 3B can be resheathed into the catheter by way of retracting the distal stabilizer 1 including the balloon 3B toward the proximal side D1 or advancing the catheter toward the distal side D1.

Next, another therapeutic procedure according to the present invention will be described. The following therapeutic procedure is for improving efficiency of thrombus suction by increasing a degree of contact between a thrombus suction catheter and a thrombus. A thrombus is an example of embolic materials. For instance, the above-described second catheter 6 or the treatment device 7 can be used as a thrombus suction catheter SC. After delivery of the thrombus suction catheter SC to a target position TP, a locking device (e.g., the locking stent 3) is caused to lock onto the inner wall V1 of a biological lumen V. A thrombus BC residing in the biological lumen V is held between the distal end of the thrombus suction catheter SC and the locking stent 3 that remains locked on the inner wall V1, and the distal end of the thrombus suction catheter SC is pressed onto the thrombus BC held between the thrombus suction catheter SC and the locking stent 3. While this state is maintained, the thrombus BC is suctioned.

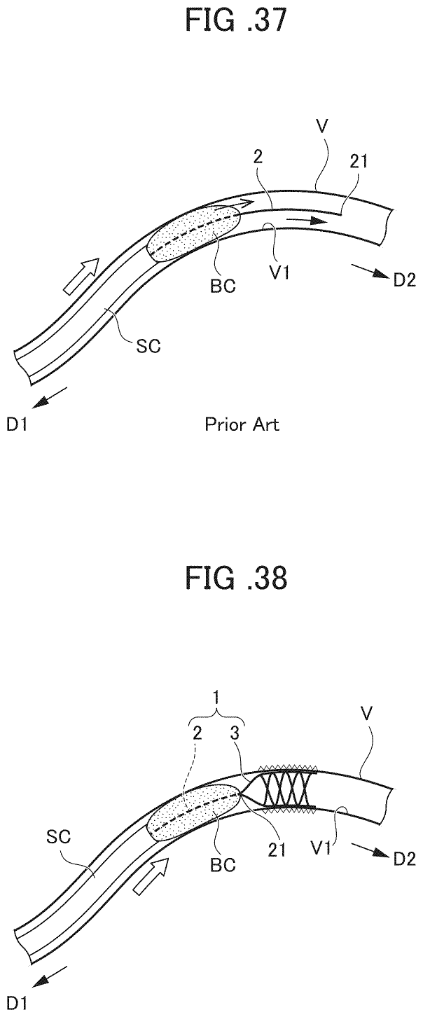

In more detail, as illustrated in FIG. 37, the thrombus BC resides in the biological lumen V at a position closer to the distal side D2 than the thrombus suction catheter SC. A delivery wire 2 passing through the thrombus suction catheter SC has its distal end 21 exposed from the distal end of the thrombus suction catheter SC and penetrating through the thrombus BC. In this state, removal of the thrombus BC out of the biological body by means of using the thrombus suction catheter SC is preferably performed by suctioning the thrombus BC while the distal end of the thrombus suction catheter SC is fully in contact with a proximal portion of the thrombus BC. However, when the proximal portion of the thrombus BC is pushed by the distal end of the thrombus suction catheter SC that is being moved toward the distal side D2, the thrombus BC will move toward the distal side D2. Therefore, the thrombus BC is easily displaced from the distal end of the thrombus suction catheter SC, making it difficult to ensure a sufficient degree of contact between the distal end of the thrombus suction catheter SC and the proximal portion of the thrombus BC. When the degree of contact between the thrombus suction catheter SC and the thrombus BC is low, the thrombus BC will be suctioned into the thrombus suction catheter SC with low efficiency.

To address this, as illustrated in FIG. 38, the distal stabilizer 1 of the present invention includes the locking stent 3 (locking device) that extends from the distal end 21 of the delivery wire 2 (thread-like delivery member) and that is capable of locking onto the inner wall V1 of the biological lumen V with its expanding force. Thus, according to the present invention, the locking stent 3 can be caused to expand at a position closer to the distal side D2 than the thrombus BC and to lock onto the inner wall V1 of the biological lumen V at the position. Note that in FIG. 38, the zigzag lines schematically indicate a region where the locking stent 3 intensively exerts its locking force. Here, suppose that the thrombus BC is likely to move toward the distal side D2 by being pushed by the distal end of the thrombus suction catheter SC that is being moved toward the distal side D2, as described above. Even in this situation, the locking stent 3 remaining locked on the inner wall V1 of the biological lumen V restricts the movement of the thrombus BC toward the distal side D2. This feature makes it easy to ensure a sufficient degree of contact between the distal end of the thrombus suction catheter SC and the proximal portion of the thrombus BC in the following way: the thrombus suction catheter SC is moved toward the distal side D2, the thrombus BC in the biological lumen V is held between the distal end of the thrombus suction catheter SC and the locking stent 3, and the distal end of the thrombus suction catheter SC is pressed onto the thrombus BC held between the thrombus suction catheter SC and the locking stent 3. The thrombus BC in this state is suctioned. In this way, suctioning is easily performed while the distal end of the thrombus suction catheter SC is fully in contact with the proximal portion of the thrombus BC. A high degree of contact between the thrombus suction catheter SC and the thrombus BC leads to a significant increase in the efficiency of suctioning of the thrombus BC into the thrombus suction catheter SC.

The therapeutic procedure illustrated in FIG. 38 may be performed using a hollow resin tube and a balloon as the thread-like delivery member and the locking device, respectively.

### EXPLANATION OF REFERENCE NUMERALS

1: Distal Stabilizer
2: Delivery Wire (Thread-like Delivery Member)
2B: Resin Tube (Thread-like Delivery Member)
21: Distal End
3, 3A: Locking Stent (Locking Device)
3B: Balloon (Locking Device)
31: Antenna
4: Body
41: Open Cell
42: Protruding Free End
43: Proximal End
45: Cell
5: First Catheter
51: Distal End
6: Second Catheter
7: Treatment Device
7A: Treatment Device
8: Third Catheter
10 Delivery System
AN: Aneurysm, Bifurcation Aneurysm
BV, BV1, BV2: Bifurcation Vessel
D1: Proximal Side
D2: Distal Side
LD: Long Axis Direction
PC: Purpose Catheter
TP: Target Position
V: Biological Lumen
V1: Inner Wall

1. A method of using a distal stabilizer for catheter delivery in a biological lumen, the distal stabilizer comprising:
a delivery member; and
a locking device that extends from a distal end of the delivery member and is configured to lock onto an inner wall of the biological lumen with an expanding force; and
the method comprising:
releasing the locking device, which has been loaded in a first catheter while being in a reduced diameter state, from a distal end of the first catheter, to cause the locking device to lock onto the inner wall;
advancing second catheter having an inner diameter larger than the first catheter toward the distal end of the delivery member while the locking device remains locked on the inner wall to deliver the second catheter in the biological lumen; and
after completion of delivering the second catheter in the biological lumen, reducing the locking device in diameter and resheathing the locking device into one of the first and second catheters, and removing the distal stabilizer out of the biological lumen.

2. The method according to claim 1, wherein
during the advancing, the second catheter is advanced by pulling the delivery member toward a proximal end of the first catheter while the locking device remains locked on the inner wall.

3. The method according to claim 1, wherein
the locking device is released by retracting the first catheter toward a proximal end of the delivery member.

4. The method according to claim 1, wherein
the locking device is configured to fit within the first catheter having an inner diameter of 0.017 inches or less.

5. The method according to claim 1, wherein
the locking device is a locking stent including a body and an antenna via which a proximal end of the body converges at the delivery member, the body having open cells with protruding free ends that protrude only toward a distal side, and
the body has a structure in which a plurality of cells are arranged helically with respect to a long axis direction of the locking stent.

6. The method according to claim 1, wherein
the locking device is at least one locking stent including a body and an antenna via which a proximal end of the body converges at the delivery member, the body having open cells with protruding free ends that protrude only toward a distal side, and
the at least one locking stent has a total effective length of 5 mm or greater.

7. The method according to claim 1, wherein
the locking device has a radiopaque portion.

8. The method according to claim 1, wherein
the locking device is a locking stent including a body and an antenna via which a proximal end of the body converges at the delivery member, the body having open cells with protruding free ends that protrude only toward a distal side, and
the locking stent has a filter that fills a gap over an entire circumferential direction of the body.

9. The method according to claim 1, wherein
the second catheter that has an inner diameter larger than the first catheter functions as a purpose catheter, and
the method further comprises:
delivering a treatment device inserted in the purpose catheter to a target position in the biological lumen by placing the purpose catheter at the target position.

10. The method according to claim 9, wherein
the target position is located in an area having a blood vessel inner diameter of from 0.5 mm to 10 mm.

11. A method for using a delivery system to deliver a treatment device in a biological lumen, the delivery system comprising:
a distal stabilizer including a delivery member and a locking device, the locking device extending from a distal end of the delivery member, and being configured to lock onto an inner wall of the biological lumen with an expanding force; and
a small catheter and a large catheter having an inner diameter larger than the small catheter; and
the method comprising:
releasing the locking device, which has been loaded in the small catheter while being in a reduced diameter state, from a distal end of the small catheter, to cause the locking device to lock onto the inner wall;
removing the small catheter out of the biological lumen while the locking device remains locked on the inner wall; and
delivering, as the treatment device, the large catheter or a device inserted in the large catheter to a target position in the biological lumen by advancing the large catheter externally fitted over the delivery member toward a distal end of the delivery member and placing the large catheter at the target position.

12. A method for using at least one delivery system to deliver a treatment device in a biological lumen, the delivery system comprising:
a distal stabilizer including a delivery member and a locking device, the locking device extending from a distal end of the delivery member, and being configured to lock onto an inner wall of the biological lumen with an expanding force; and
a small catheter and a large catheter having an inner diameter larger than the small catheter; and
the method comprising:
releasing, at a site distal to an aneurysm, the locking device, which has been loaded in the small catheter while being in a reduced diameter state, from a distal end of the small catheter, to cause the locking device to lock onto the inner wall; and
advancing the purpose catheter fitted over the delivery member toward the distal side and placing the purpose catheter at the target position in the biological lumen to deliver, as the treatment device, the large catheter or a device inserted in the large catheter to a target position in the biological lumen,
wherein the target position is the aneurysm or a position distal to the aneurysm.

13. The method according to claim 12, wherein
the aneurysm is a bifurcation aneurysm.

14. The method according to claim 13, wherein
the at least one delivery system comprises a plurality of delivery systems for use in combination with each other,
each of the delivery systems is configured to lock onto an inner wall of an associated one of bifurcation vessels between which the bifurcation aneurysm resides, and
each of the delivery systems is configured to deliver the treatment device as a flow diverter and to place the treatment device over an area from a location proximal to the bifurcation aneurysm to the bifurcation vessel.

15. The method according to claim 11, wherein
the locking device is a locking stent, and
the locking stent includes at least one of closed cells and open cells.

16. The method according to claim 9, wherein
the locking device is a locking stent including a body and an antenna via which a proximal end of the body converges at the delivery member, the body having open cells with protruding free ends that protrude only toward a distal side, and
the locking stent has a filter that fills a gap over an entire circumferential direction of the body.

17. The method according to claim 9, wherein
the treatment device is a thrombus suction device, a flow diverter, an aneurysm embolization device, a thrombus removal device, an aneurysm treatment stent, an intracranial artery stenosis treatment stent, a balloon catheter, a shunt, or a device configured to release a liquid embolic material.

18. The method according to claim 9, wherein
the treatment device is a suction catheter; and
the method further comprising:
after delivery of the treatment device to the target position
locking the locking device onto an inner wall of the biological lumen, and holding an embolic material in the biological lumen between a distal end of the suction catheter and the locking device remaining locked on the inner wall; and
pressing a distal end of the suction catheter onto the embolic material held between the distal end of the suction catheter and the locking device, and suctioning the embolic material by the suction catheter while the embolic material is held between the distal end of the suction catheter pressed onto the embolic material and the locking device remaining locked on the inner wall.

19. The method according to claim 2, wherein
the locking device is released by retracting the first catheter toward a proximal end of the delivery member.

20. The method according to claim 2, wherein
the locking device is configured to fit within the first catheter having an inner diameter of 0.017 inches or less.
